(19) United States
(12) Patent Application Publication
(10) Pub. No.: US 2010/0233177 A1
(43) Pub. Date: Sep. 16, 2010

(54) MOLECULES AND METHODS FOR MODULATING PROPROTEIN CONVERTASE SUBTILISIN/KEXIN TYPE 9 (PCSK9)

(76) Inventors: David Langdon Yowe, Cambridge, MA (US); Dmitri Mikhailov, Cambridge, MA (US); Tony Fleming, Cambridge, MA (US)

Correspondence Address: Charles E. Lykes, Jr., Charles E. Lykes, Jr. Attorney At Law, 501 South Fort Harrison Avenue Suite 101, Clearwater, FL, 33756-5348, US

(21) Appl. No.: 12/595,538

(22) PCT Filed: Apr. 11, 2008
(86) PCT No.: PCT/EP08/54417

§ 371 Date: Oct. 12, 2009

Related U.S. Application Data
(60) Provisional application No. 60/911,654, filed on Apr. 13, 2007.

(51) Int. Cl. A61K 39/395; C07K 16/40; C12N 5/02; A61P 9/00

(52) U.S. Cl. 424/139.1; 530/387.9; 530/387.3; 435/375; 530/327; 530/326; 530/329

(57) ABSTRACT

Epitopes of Proprotein convertase subtilisin/kexin type 9 (PCSK9), compositions that bind to PCSK9 and PCSK9 epitopes, and methods of using the compositions are described herein.

### TECHNICAL FIELD

This invention relates to antigen binding molecules, epitopes bound by those molecules, and methods of using the molecules.

### BACKGROUND

Proprotein convertase subtilisin/kexin type 9 (PCSK9) (also known as Neural apoptosis-regulated convertase 1, or NARC-1) is a member of the proteinase K secretory subtilisin-like subfamily of serine proteases (Naureckiene et al., 2003 Arc. Biochem. Biophys. 420:55-67). Human PCSK9 is a secreted protein expressed primarily in the kidneys, liver and intestines. It has a three domains: an inhibitory pro-domain (amino acids 1-152; including a signal sequence at amino acids 1-30), a serine protease domain (amino acids 153-448), and a C-terminal domain 210 residues in length (amino acids 449-692), which is rich in cysteine residues. PCSK9 is synthesized as a zymogen that undergoes autocatalytic cleavage between the pro-domain and catalytic domain in the endoplasmic reticulum. The pro-domain remains bound to the mature protein after cleavage, and the complex is secreted. The cysteine-rich domain may play a role analogous to the P-(processing) domains of other Furin/Kexin/Subtilisin-like serine proteases, which appear to be essential for folding and regulation of the activated protease. Mutations in PCSK9 are associated with abnormal levels of low density lipoprotein cholesterol (LDL-c) in the blood plasma (Horton et al., 2006 Trends. Biochem. Sci. 32(2):71-77).

### SUMMARY

The present invention relates to epitopes of PCSK9, PCSK9 binding molecules, and methods of using the molecules. PCSK9 binding molecules interact with PCSK9 and modulate PCSK9 functions. PCSK9 binding molecules can be used to increase LDL-receptor (LDL-R) levels and reduce cholesterol levels.

In various aspects, the invention provides PCSK9 binding molecules that modulate (e.g., inhibit) one or more biological functions of PCSK9. For example, a PCSK9 binding molecule can inhibit proteolytic activity of PCSK9 (e.g., proteolysis of the PCSK9 pro-domain) and/or an interaction between PCSK9 and a PCSK9 receptor (e.g., PCSK9 binding to LDL-R). PCSK9 downregulates LDL-R in a post-transcriptional manner. Thus, inhibition of PCSK9 results in increased LDL-R levels. Increased levels of LDL-R in vivo allows for increased LDL-R mediated uptake of LDL-c. Thus, binding molecules that interfere with PCSK9 regulation of LDL-R ultimately reduce levels of circulating LDL-c.

PCSK9 binding molecules include, for example, antibodies that bind to PCSK9 (e.g., within a particular domain or epitope of PCSK9, such as the catalytic domain or the cysteine-rich domain), and polypeptides that include antigen binding portions of such antibodies. PCSK9 binding molecules also include molecules in which the binding portion is not derived from an antibody, e.g., PCSK9 binding molecules derived from polypeptides that have an immunoglobulin-like fold, and in which the antigen binding portion is engineered to bind PCSK9 through randomization, selection, and affinity maturation.

Accordingly, in one aspect, the invention features a PCSK9 binding molecule including an antigen binding portion of an antibody that binds (e.g., specifically binds) to a PCSK9, wherein the antigen binding portion binds to an epitope within the catalytic domain of human PCSK9 (SEQ ID NO:1) within or overlapping (e.g., comprising or consisting of all or a portion) one of the following: (a) amino acids 166-177 of SEQ ID NO:1 (i.e., an epitope within or overlapping the following sequence: YRADEYQPPDGG (SEQ ID NO:4)); (b) amino acids 187-202 of SEQ ID NO:1 (i.e., an epitope within or overlapping the following sequence: TSIQSDHREIEGRVMV (SEQ ID NO:5)); (c) amino acids 206-219 of SEQ ID NO:1 (i.e., an epitope within or overlapping the following sequence: ENVPEEDGTRFHRQ (SEQ ID NO:6)); (d) amino acids 231-246 of SEQ ID NO:1 (i.e., an epitope within or overlapping the following sequence: AGVVSGRDAGVAKGAS (SEQ ID NO:7)); (e) amino acids 277-283 of SEQ ID NO:1 (i.e., an epitope within or overlapping the following sequence: VQPVGPL (SEQ ID NO:8)); (f) amino acids 336-349 of SEQ ID NO:1 (i.e., an epitope within or overlapping the following sequence: VGATNAQDQPVTLG (SEQ ID NO:9)); (g) amino acids 368-383 of SEQ ID NO:1 (i.e., an epitope within or overlapping the following sequence: IIGASSDCSTCFVSQS (SEQ ID NO:10)); or (h) amino acids 426-439 of SEQ ID NO:1 (i.e., an epitope within or overlapping the following sequence: EAWFPEDQRVLTPN (SEQ ID NO:11)).

For example, the antigen binding portion binds to an epitope within amino acids 166-171, 169-174, or 172-177 of SEQ ID NO:1; the antigen binding portion binds to an epitope within amino acids 187-193, 191-196, 194-199, or 197-202 of SEQ ID NO:1; the antigen binding portion binds to an epitope within amino acids 206-211, 209-214, 212-217, 215-219 of SEQ ID NO:1; the antigen binding portion binds to an epitope within amino acids 231-237, 235-240, 238-243, 241-246 of SEQ ID NO:1; the antigen binding portion binds to an epitope within amino acids 277-282, or 279-283 of SEQ ID NO:1; the antigen binding portion binds to an epitope within amino acids 336-341, 339-343, 341-346, or 344-349 of SEQ ID NO:1; the antigen binding portion binds to an epitope within amino acids 368-374, 372-377, 375-380, or 378-383 of SEQ ID NO:1; the antigen binding portion binds to an epitope within amino acids 426-431, 429-434, 432-437, or 435-439 of SEQ ID NO:1).

In another aspect, the invention features an isolated PCSK9 binding molecule comprising an antigen binding portion of an antibody that binds (e.g., specifically binds) to a PCSK9, wherein the antigen binding portion binds to an epitope within the cysteine-rich domain of human PCSK9 within or overlapping one of the following: (a) amino acids 443-500 of SEQ ID NO: 1; (b) amino acids 557-590; or (c) amino acids 636-678.

In various embodiments, the antigen binding portion specifically binds to an epitope of human PCSK9 within or overlapping within or overlapping one of the following: (a) amino acids 443-458 of SEQ ID NO:1 (i.e., an epitope within or overlapping the following sequence: ALPPSTHGAGWQLFCR (SEQ ID NO:12)); (b) amino acids 459-476 of SEQ ID NO:1 (i.e., an epitope within or overlapping the following sequence: TVWSAHSGPTRMATAIAR (SEQ ID NO:13)); (c) amino acids 486-500 of SEQ ID NO:1 (i.e., an epitope within or overlapping the following sequence: CSSFSRSGKRRGERM (SEQ ID NO:14)); (d) amino acids 557-573 of SEQ ID NO:1 (i.e., an epitope within or overlapping the following sequence: HVLTGCSSHWEVEDLGT (SEQ ID NO:15)); (e) amino acids 577-590 of SEQ ID NO:1 (i.e., an epitope within or overlapping the following sequence: PVLRPRGQPNQCVG (SEQ ID NO:16)); (f) amino acids 636-645 of SEQ ID NO:1 (i.e., an epitope within or overlapping the following sequence: SALPGTSHVL (SEQ ID NO:17)); (g) amino acids 659-677 of SEQ ID NO:1 (i.e., an epitope within or overlapping the following sequence: RDVSTTGSTSEEAVTAVAI (SEQ ID NO:18));

For example, the antigen binding portion binds to an epitope within amino acids 443-449, 447-452, 450-455, 453-458 of SEQ ID NO:1; the antigen binding portion binds to an epitope within amino acids 459-465, 463-468, 466-471, 469-474, or 472-476 of SEQ ID NO:1; the antigen binding portion binds to an epitope within amino acids 486-491, 489-494, 492-497, or 495-500 of SEQ ID NO:1; the antigen binding portion binds to an epitope within amino acids 557-563, 561-566, 564-569, 567-572, or 569-573 of SEQ ID NO:1; the antigen binding portion binds to an epitope within amino acids 577-582, 580-585, 583-588, or 586-590 of SEQ ID NO:1; the antigen binding portion binds to an epitope within amino acids 636-643, or 640-645 of SEQ ID NO:1; the antigen binding portion binds to an epitope within amino acids 659-665, 663-668, 665-670, 668-673, or 671-677 of SEQ ID NO:1.

In another aspect, the invention features an isolated PCSK9 binding molecule including an antigen binding portion of an antibody that binds (e.g., specifically binds) to a PCSK9, wherein the antigen binding portion binds to an epitope within the pro-domain of human PCSK9 within or overlapping amino acids 89-134 of SEQ ID NO:1.

In various embodiments, the antigen binding portion specifically binds to an epitope of human PCSK9 within or overlapping one of the following: (a) amino acids 89-101 of SEQ ID NO:1 (i.e., an epitope within or overlapping the following sequence: SQSERTARRLQAQ (SEQ ID NO:2)); or (b) amino acids 106-134 of SEQ ID NO:1 (i.e., an epitope within or overlapping the following sequence: GYLTKILHVFHGLLPGFLVKMSGDLLELA (SEQ ID NO:3)). For example, the antigen binding portion specifically binds to an epitope within amino acids 123-131 of SEQ ID NO:1.

For example, the antigen binding portion binds to an epitope within amino acids 89-94, 92-97, or 95-101 of SEQ ID NO:1; the antigen binding portion binds to an epitope within amino acids 106-111, 109-114, 112-117, 115-120, 118-123, 121-126, 124-129, or 127-134 of SEQ ID NO:1.

In a particular embodiment, the antigen binding portion specifically binds to an epitope within the pro-domain of human PCSK9 within or overlapping amino acids 101-107 of SEQ ID NO:1 (amino acids QAARRGY).

In another embodiment, the antigen binding portion specifically binds to an epitope within the pro-domain of human PCSK9 within or overlapping amino acids 123-132 of SEQ ID NO:1 (amino acids LVKMSGDLLE). These amino acids fall within amino acids 106-134 of the pro-domain of PCSK9 (SEQ ID NO:3; GYLTKILHVFHGLLPGFLVKMSGDLLELA).

In another embodiment, the antigen binding portion specifically binds to PCSK9 with amino acids 101-132 of SEQ ID NO:1 (i.e., binds to an epitope within SEQ ID NO:2, an epitope within SEQ ID NO:3, or an epitope that overlaps SEQ ID NOs: 2 and 3, i.e., includes at least one amino acid from SEQ ID NO:2 and SEQ ID NO:3).

In another embodiment, the antigen binding portion specifically binds to PCSK9 within amino acids 101-132 of SEQ ID NO:1 and comprises at least one amino acid from SEQ ID NO:2 (e.g., glutamine) and at least one amino acid from SEQ ID NO:3 (e.g., glycine and/or tyrosine).

In another embodiment, the antigen binding portion specifically binds to PCSK9 within amino acids 101-132 of SEQ ID NO:1 and comprises at least one amino acid from SEQ ID NO:2 (e.g., glutamine) and at least one amino acid from SEQ ID NO:3 (e.g., glycine and/or tyrosine).

In another embodiment, the antigen binding portion specifically binds to PCSK9 at an epitope that overlaps at least one amino acid from SEQ ID NO:2 (e.g., glutamine) and at least one amino acid from SEQ ID NO:3 (e.g., glycine and/or tyrosine).

In another aspect, the invention features an isolated PCSK9 binding molecule that cross-competes for binding with any of the aforementioned PCSK9 binding molecules. Accordingly, such cross-competing binding molecules can, for example, interfere with binding (e.g., of an antibody or other PCSK9 binding molecule comprising an antigen binding portion of an antibody that binds) to amino acids 101-107 or 123-132 of SEQ ID NO:1 by binding to spatially proximate epitopes.

In various embodiments, the PCSK9 binding molecule (e.g., the PCSK9 binding molecule that binds to an epitope within the catalytic domain, within the cysteine-rich domain, or within the pro-domain) is cross reactive with a PCSK9 of a non-human primate (e.g., a cynomolgus monkey, or a rhesus monkey). In various embodiments, the antigen binding portion is cross reactive with a PCSK9 of a rodent species (e.g., murine PCSK9, rat PCSK9).

In various embodiments, the antigen binding portion binds to a linear epitope.

In various embodiments, the antigen binding portion binds to a non-linear epitope. In one example, the antigen binding portion binds to a non-linear epitope including, or consisting of, at least one portion of each of the following linear epitopes: (a) amino acids 89-101 of SEQ ID NO:1; and (b) amino acids 106-134 of SEQ ID NO:1. In another example, the antigen binding portion binds to a non-linear epitope including, or consisting of, at least one portion of each of the following linear epitopes: (a) amino acids 166-177 of SEQ ID NO:1; and (b) amino acids 443-458 of SEQ ID NO:1. In yet another example, the antigen binding portion binds to a non-linear epitope including, or consisting of, at least one portion of two or three of the following linear epitopes: (a) amino acids 187-202 of SEQ ID NO: 1; (b) amino acids 231-246 of SEQ ID NO:1; and (c) amino acids 368-383 of SEQ ID NO:1. In another example, the antigen binding portion binds to a non-linear epitope including, or consisting of, at least one portion of each of the following linear epitopes: (a) amino acids 206-219 of SEQ ID NO:1; and (b) amino acids 277-283 of SEQ ID NO:1. In another example, the antigen binding portion binds to a non-linear epitope including, or consisting of, at least one portion of each of the following linear epitopes: (a) amino acids 336-349 of SEQ ID NO:1; and (b) amino acids 426-439 of SEQ ID NO:1. In another example, the antigen binding portion binds to a non-linear epitope including, or consisting of, at least one portion of two or three of the following linear epitopes: (a) amino acids 459-476 of SEQ ID NO:1; (b) amino acids 486-500 of SEQ ID NO:1; and (c) amino acids 557-573 of SEQ ID NO:1. In another example, the antigen binding portion binds to a non-linear epitope including, or consisting of, at least one portion of two or three of the following linear epitopes: (a) amino acids 577-590 of SEQ ID NO:1; (b) amino acids 636-645 of SEQ ID NO:1; and (c) amino acids 659-677 of SEQ ID NO:1.

In a particular embodiment, the antigen binding portion binds to a non-linear epitope (e.g., a conformational epitope) comprising all or a portion of (a) amino acids 101-107 of SEQ ID NO:1; and (b) amino acids 123-132 of SEQ ID NO:1.

In various embodiments, the efficacy of binding of the PCSK9 binding molecules correlates to the location of binding within a particular domain or epitope of PCSK9.

In various embodiments, the antigen binding portion of the PCSK9 binding molecule binds to PCSK9 with a dissociation constant (K_{D}) equal to or less than 10 nM, 1 nM, 0.5 nM, 0.25 nM, or 0.1 nM.

In various embodiments, the antigen binding portion of the PCSK9 binding molecule binds to PCSK9 of a non-human primate (e.g., cynomolgus monkey or chimpanzee) with a K_{D} equal to or less than 0.3 nM.

In various embodiments, antigen binding portion binds to mouse PCSK9 with a K_{D} equal to or less than 0.5 nM.

The antibody can be a chimeric (e.g., humanized) antibody or a human antibody, or a humaneered antibody.

In one embodiment, the antigen binding portion is an antigen binding portion of a human antibody.

The antigen binding portion can be an antigen binding portion of a monoclonal antibody or a polyclonal antibody.

The PCSK9 binding molecule includes, for example, an Fab fragment, an Fab′ fragment, an F(ab′)₂, or an Fv fragment of the antibody.

In one embodiment, the PCSK9 binding molecule is a human antibody.

In one embodiment, the PCSK9 binding molecule includes a single chain Fv.

In one embodiment, the PCSK9 binding molecule includes a diabody (e.g., a single chain diabody, or a diabody having two polypeptide chains).

In some embodiments, the antigen binding portion of the antibody is derived from an antibody of one of the following isotypes: IgG1, IgG2, IgG3 or IgG4. In some embodiments, the antigen binding portion of the antibody is derived from an antibody of an IgA or IgE isotype.

The PCSK9 binding molecule (e.g., the PCSK9 binding molecule that binds to an epitope within the catalytic domain, within the cysteine-rich domain, or within the pro-domain) can exhibit one or more of a number of biological activities. In various embodiments, the PCSK9 binding molecule inhibits PCSK9 binding to a PCSK9 ligand. In some embodiments, the PCSK9 binding molecule inhibits binding to the PCSK9 ligand at pH 7-8. In some embodiments, the PCSK9 binding molecule inhibits binding at a pH below pH 7 (e.g., at pH 5-7). For example, the PCSK9 binding molecule inhibits PCSK9 binding to the PCSK9 ligand by at least 5%, 10%, 15%, 25%, or 50%, relative to a control (e.g., relative to binding in the absence of the PCSK9 binding molecule).

For example, the PCSK9 binding molecule can inhibit PCSK9 binding to a low density lipoprotein receptor (LDL-R) (e.g., the PCSK9 binding molecule inhibits PCSK9 binding to LDL-R at pH 7 and lower pH, e.g., pH 5-7).

The PCSK9 pro-domain is cleaved from, and remains non-covalently associated with, the mature PCSK9 polypeptide. In one embodiment, a PCSK9 binding molecule competes with a PCSK9 pro-domain for binding to the catalytic or cysteine-rich domain (or vice versa), and inhibits a biological activity of PCSK9.

In some embodiments, the PCSK9 binding molecule inhibits proteolytic activity of PCSK9 (e.g., proteolysis of the PCSK9 pro-domain, or of another PCSK9 substrate). For example, the PCSK9 binding molecule inhibits PCSK9 proteolytic activity by at least 5%, 10%, 15%, 25%, or 50%, relative to a control (e.g., relative to activity in the absence of the PCSK9 binding molecule).

In some embodiments, the PCSK9 binding molecule inhibits a PCSK9-dependent decrease of LDL-R (e.g., PCSK9 dependent degradation of LDL-R) on a cell, e.g., a hepatocyte. For example, the PCSK9 binding molecule inhibits a PCSK9-dependent decrease of LDL-R by at least 5%, 10%, 15%, 25%, or 50%, relative to a control (e.g., relative to the decrease of LDL-R in the absence of the PCSK9 binding molecule). In these embodiments, an increase in LDL-R levels indicates that the PCSK9 binding molecule inhibits the PCSK9-dependent decrease of LDL-R.

In certain embodiments, a PCSK9 binding molecule, when contacted with a cell, e.g., a hepatocyte under conditions in which PCSK9 is present, increases LDL-c uptake by the hepatocyte, relative to LDL-c uptake by a hepatocyte in the absence of the PCSK9 binding molecule. For example, the PCSK9 binding molecule increases LDL-c uptake by at least 5%, 10%, 15%, 25%, or 50%, relative to a control (e.g., relative to binding in the absence of the PCSK9 binding molecule).

The PCSK9 binding molecule can bind to PCSK9 in the presence of LDL-c and/or it can bind to PCSK9 in the presence of serum (e.g., in the presence of at least 1%, 5%, 10%, 25%, 50%, serum).

The invention also features non-antibody PCSK9 binding molecules. A non-antibody PCSK9 binding molecule includes a PCSK9 binding domain that has an amino acid sequence derived from an immunoglobulin-like (Ig-like) fold of a non-antibody polypeptide, such as one of the following: tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin. In general, the amino acid sequence of the PCSK9 binding domain is altered, relative to the amino acid sequence of the immunoglobulin-like fold, such that the PCSK9 binding domain specifically binds to the PCSK9 (i.e., wherein the immunoglobulin-like fold does not specifically bind to the PCSK9).

In various embodiments, the amino acid sequence of the PCSK9 binding domain is at least 60% identical (e.g., at least 65%, 75%, 80%, 85%, or 90% identical) to an amino acid sequence of an immunoglobulin-like fold of a fibronectin, a cytokine receptor, or a cadherin.

In various embodiments, the amino acid sequence of the PCSK9 binding domain is at least 60%, 65%, 75%, 80%, 85%, or 90% identical to an amino acid sequence of an immunoglobulin-like fold of one of the following: tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin.

In various embodiments, the PCSK9 binding domain binds to the PCSK9 with a K_{D} equal to or less than 10 nM (e.g., 1 nM, 0.5 nM, 0.1 nM).

In some embodiments, the Ig-like fold is an Ig-like fold of a fibronectin, e.g., an Ig-like fold of fibronectin type III (e.g., an Ig-like fold of module 10 of fibronectin III).

The invention also provides peptides corresponding to antigenic epitopes of PCSK9. In one aspect, the invention features a peptide consisting of an amino acid sequence at least 90% identical to one of following amino acid sequences:

| | |
|---|---|
| YRADEYQPPDGG; | (SEQ ID NO: 4) |
| TSIQSDHREIEGRVMV; | (SEQ ID NO: 5) |
| ENVPEEDGTRFHRQ; | (SEQ ID NO: 6) |
| AGVVSGRDAGVAKGAS; | (SEQ ID NO: 7) |
| VQPVGPL; | (SEQ ID NO: 8) |
| VGATNAQDQPVTLG; | (SEQ ID NO: 9) |
| IIGASSDCSTCFVSQS; | (SEQ ID NO: 10) |
| EAWFPEDQRVLTPN; | (SEQ ID NO: 11) |
| ALPPSTHGAGWQLFCR; | (SEQ ID NO: 12) |
| TVWSAHSGPTRMATAIAR; | (SEQ ID NO: 13) |
| CSSFSRSGKRRGERM; | (SEQ ID NO: 14) |
| HVLTGCSSHWEVEDLGT; | (SEQ ID NO: 15) |
| PVLRPRGQPNQCVG; | (SEQ ID NO: 16) |
| SALPGTSHVL; | (SEQ ID NO: 17) |
| RDVSTTGSTSEEAVTAVAI; | (SEQ ID NO: 18) |
| SQSERTARRLQAQ; | (SEQ ID NO: 2) |
| or | |
| GYLTKILHVFHGLLPGFLVKMSGDLLELA. | (SEQ ID NO: 3) |

In another aspect, the invention provides compositions for eliciting antibodies that specifically bind to PCSK9 when the composition is administered to an animal. The compositions include, for example, one of the following peptides: YRADEYQPPDGG (SEQ ID NO:4); TSIQSDHREIEGRVMV (SEQ ID NO:5); ENVPEEDGTRFHRQ (SEQ ID NO:6); AGVVSGRDAGVAKGAS (SEQ ID NO:7); VQPVGPL (SEQ ID NO:8); VGATNAQDQPVTLG (SEQ ID NO:9); IIGASSDCSTCFVSQS (SEQ ID NO:10); EAWFPEDQRVLTPN (SEQ ID NO:11); ALPPSTHGAGWQLFCR (SEQ ID NO:12); TVWSAHSGPTRMATAIAR (SEQ ID NO:13); CSSFSRSGKRRGERM (SEQ ID NO:14); HVLTGCSSHWEVEDLGT (SEQ ID NO:15); PVLRPRGQPNQCVG (SEQ ID NO:16); SALPGTSHVL (SEQ ID NO:17) RDVSTTGSTSEEAVTAVAI (SEQ ID NO:18); SQSERTARRLQAQ (SEQ ID NO:2); GYLTKILHVFHGLLPGFLVKMSGDLLELA (SEQ ID NO:3); a peptide thereof with less than 5 amino acid changes; or a fragment thereof (e.g., fragments containing 5, 6, 7, 8, 9, 10, 11, or 12 amino acids). The peptide can be modified to increase antigenicity, e.g., by coupling to a carrier protein.

The invention also features a pharmaceutical composition that includes a PCSK9 binding molecule described herein. The composition includes, for example, a PCSK9 binding molecule and a pharmaceutically acceptable carrier.

The invention also features methods of using the PCSK9 binding molecules described herein.

For example, in one aspect, the invention features a method of increasing LDL-R levels on a cell, e.g., a hepatocyte. The method includes contacting the hepatocyte with a PCSK9 binding molecule (e.g., a PCSK9 binding molecule including an antigen binding portion of an antibody that specifically binds to a PCSK9), thereby reducing downregulation of LDL-R by PCSK9 and increasing LDL-R levels on the hepatocyte.

In another aspect, the invention features a method of increasing LDL-c uptake by a cell, e.g., a hepatocyte. The method includes contacting the hepatocyte with a PCSK9 binding molecule (e.g., a PCSK9 binding molecule including an antigen binding portion of an antibody that specifically binds to a PCSK9), thereby reducing downregulation of LDL-R by PCSK9 and increasing LDL-c uptake by the hepatocyte.

In another aspect, the invention features a method of modulating PCSK9 activity in a subject. The method includes administering to the subject a PCSK9 binding molecule (e.g., a PCSK9 binding molecule including an antigen binding portion of an antibody that specifically binds to a PCSK9) that modulates a biological activity of the PCSK9. The PCSK9 binding molecule exhibits one or more of the following activities: (a) inhibiting PCSK9 binding to a LDL-R; (b) inhibiting proteolytic activity of the PCSK9; (c) inhibiting PCSK9 dependent decrease of LDL-R on a hepatocyte; and (d) inhibiting PCSK9 dependent degradation of LDL-R in hepatocyte cells.

In another aspect, the invention features a method of reducing plasma cholesterol in a subject. The method includes administering to the subject a pharmaceutical composition including a PCSK9 binding molecule described herein in an amount effective to reduce plasma cholesterol in the subject. The amount can be an amount effective to reduce LDL-c. The subject's concentration of plasma LDL-c can be reduced by at least 5%, relative to plasma LDL-c prior to administering the composition (e.g., the concentration of plasma LDL-c is reduced by at least 10%, 15%, or 20%). In some embodiments, the subject is also receiving therapy with a second cholesterol-reducing agent, such as a statin.

In various embodiments, the subject has, or is at risk for, a lipid disorder (e.g., hyperlipidemia, type I, type II, type III, type IV, or type V hyperlipidemia, secondary hypertriglyceridemia, hypercholesterolemia, xanthomatosis, cholesterol acetyltransferase deficiency). For example, the subject is hypercholesterolemic or is at risk for hypercholesterolemia; the subject has, or is at risk for, atherosclerosis; the subject has, or is at risk for, a cardiovascular disorder.

In some embodiments, the subject is statin-intolerant (e.g., the subject suffers from adverse side effects when taking a statin drug), and/or the subject is resistant to statin therapy. (e.g., statin therapy did not cause cholesterol reduction in the subject).

In some embodiments, the subject's total plasma cholesterol level is 200 mg/dl or greater, prior to administration of the composition.

In some embodiments, the subject's plasma LDL-c level is 160 mg/dl or greater, prior to administration of the composition.

In some embodiments, the composition is administered intravenously.

In some embodiments, the PSCK9 binding molecules can be used to prepare a medicament for the treatment of disease associated with high cholesterol levels.

The details of one or more embodiments of the invention are set forth in the accompanying drawings and the description below. Other features, objects, and advantages of the invention will be apparent from the description and drawing, and from the claims.

### DESCRIPTION OF DRAWINGS

FIG. 1 is a schematic diagram of human PCSK9, indicating the location of the signal peptide, pro-domain, catalytic domain, and C-terminal (cysteine-rich) domain in the linear sequence.

FIG. 2 is a depiction of a three-dimensional structural model of human PCSK9. The numbers indicate the location of epitopes listed in Table 2.

FIG. 3 depicts the results of the Biacore binding affinity studies of H1-Fab binding to hPCSK9. The sensograms (jagged black lines) are the binding curves of H1-Fab at concentrations of 0.78, 1.56, 3.12, 6.25. and 12.5 nM. The 1:1 global fit (smooth black lines) gives the following binding parameters: κ_{d}=3.41×10⁻³ (1/s), κₐ=3.23×10⁵ (1/Ms), and K_{D}=1.05 and 10⁴ (M).

FIGS. 4A-C illustrate that H1-Fab can (4A) disrupt the hPCSK9/LDL-R interaction and lead to (4B) increased surface LDL-R levels and (4C) increased LDL-uptake by HepG2 cells.

FIGS. 5A-C depict the fluid connection scheme of an automated deuterium exchange mass spectrometry (DXMS) system. Valve positions for the loading/inline proteolysis phase, desalting stage, and separation stage of the experiment are illustrated in FIGS. 5A, 5B, and 5C respectively.

FIG. 6 is a schematic depicting the complementary Hydrogen/Deuterium (H/D) exchange experiments (i.e., protection, control and In-D₂O experiments) and expected outcomes.

FIGS. 7A-B depict the observed change in deuteration as a function of residue number of hPCSK9 for (A) the protection experiments and (B) the In-D₂O experiments performed on hPCSK9 and hPCSK9:H1-Fab complex.

FIG. 8 depicts the cartoon crystal structure of hPCSK9 with the two amino acid stretches (i.e., amino acid residues 101-107 (QAARRGY) and 123-132 (LVKMSGDLLE)) predicted to form a non-linear epitope.

### DETAILED DESCRIPTION

The present invention provides molecules that bind to PCSK9 (“PCSK9 binding molecules”), particularly human antibodies and portions thereof that bind to human PCSK9 and modulate its functions. Epitopes of PCSK9 and agents that bind these epitopes are also provided herein.

The full length sequence of human PCSK9 (hPCSK9) is found under Genbank® Accession Number GI:119627065, gb|EAX06660.1, and is shown in Table 1 as SEQ ID NO: 1. An mRNA sequence encoding hPCSK9 is found under Accession Number GI:31317306, NM_{—}174936.

TABLE 1
Human PCSK9 Amino Acid Sequence

(SEQ ID NO: 1)

MGTVSSRRSWWPLPLLLLLLLLLGPAGARAQEDEDGDYEELVLALRSEED
GLAEAPEHGTTATFHRCAKDPWRLPGTYVVVLKEETHLSQSERTARRLQA
QAARRGYLTKILHVFHGLLPGFLVKMSGDLLELALKLPHVDYIEEDSSVF
AQSIPWNLERITPPRYRADEYQPPDGGSLVEVYLLDTSIQSDHREIEGRV
MVTDFENVPEEDGTRFHRQASKCDSHGTHLAGVVSGRDAGVAKGASMRSL
RVLNCQGKGTVSGTLIGLEFIRKSQLVQPVGPLVVLLPLAGGYSRVLNAA
CQRLARAGVVLVTAAGNFRDDACLYSPASAPEVITVGATNAQDQPVTLGT
LGTNFGRCVDLFAPGEDIIGASSDCSTCFVSQSGTSQAAAHVAGIAAMML
SAEPELTLAELRQRLIHFSAKDVINEAWFPEDQRVLTPNLVAALPPSTHG
AGWQLFCRTVWSAHSGPTRMATAIARCAPDEELLSCSSFSRSGKRRGERM
EAQGGKLVCRAHNAFGGEGVYAIARCCLLPQANCSVHTAPPAEASMGTRV
HCHQQGHVLTGCSSHWEVEDLGTHKPPVLRPRGQPNQCVGHREASIHASC
CHAPGLECKVKEHGIPAPQEQVTVACEEGWTLTGCSALPGTSHVLGAYAV
DNTCVVRSRDVSTTGSTSEEAVTAVAICCRSRHLAQASQELQ

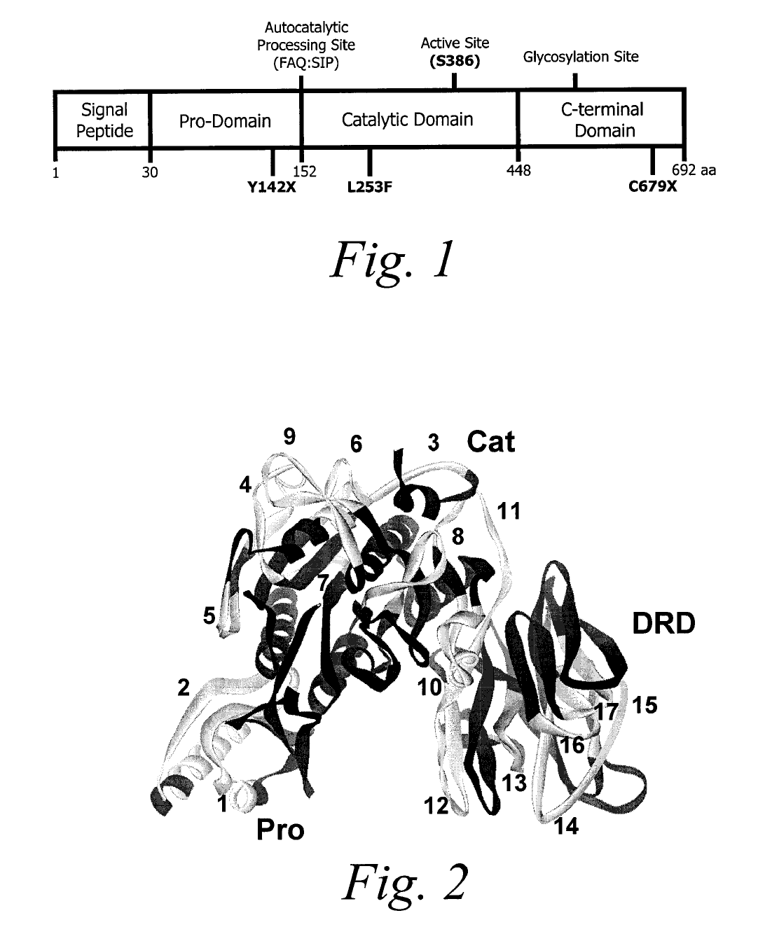

The locations of the signal peptide, pro-domain, catalytic domain, and C-terminal cysteine rich domain in the linear sequence of SEQ ID NO:1 are shown in FIG. 1. Human PCSK9 is N-glycosylated at N533. It is sulfated at Y53 and in the catalytic (protease) domain. The concentration of hPCSK9 in human plasma ranges from 50-600 ng/ml (Lagace et al., 2006 J. Clin. Inv. 116(11):2995-3005). Certain mutations in hPCSK9 are associated with elevated and reduced plasma levels of LDL-c (Horton et al., 2006 Trends. Biochem. Sci. 32(2):71-77). The following mutations are associated with elevated LDL-c: S127R, F216L, D374Y, N425S, and R496W. The following mutations are associated with reduced LDL-c: R46L, AR97, G106R, Y142X, L253F, A443T, and C679X.

Predicted chimpanzee PCSK9 amino acid sequences are found in Genbank® under Acc. No. GI:114556790, XP_{—}001154126; and Acc. No. GI:114556788, XP_{—}513430. The amino acid sequence of mouse PCSK9 is found under Acc. No. GI:23956352, NP_{—}705793. The rat PCSK9 amino acid sequence is found under Acc. No. GI:77020250, NP_{—}954862. The amino acid sequence of hPCSK9 is 98.7% identical to chimpanzee PCSK9, 79.5% identical to rat PCSK9, and 78.9% identical mouse PCSK9.

The amino acid sequences of antigenic epitopes of hPCSK9 and their position within the hPCSK9 sequence of SEQ ID NO:1 are listed in Table 2.

TABLE 2
Antigenic epitopes of hPCSK9

| | | SEQ | | |
|---|---|---|---|---|
| # | amino acid sequence | ID NO: | domain | position |
| 1 | SQSERTARRLQAQ | 2 | Pro | 89-101 |
| 2 | GYLTKILHVFHGLLPGFLVKMSGD | 3 | Pro | 106-134 |
| | LLELA | | | |
| 3 | YRADEYQPPDGG | 4 | Cat | 166-177 |
| 4 | TSIQSDHREIEGRVMV | 5 | Cat | 187-202 |
| 5 | ENVPEEDGTRFHRQ | 6 | Cat | 206-219 |
| 6 | AGVVSGRDAGVAKGAS | 7 | Cat | 231-246 |
| 7 | VQPVGPL | 8 | Cat | 277-283 |
| 8 | VGATNAQDQPVTLG | 9 | Cat | 336-349 |
| 9 | IIGASSDCSTCFVSQS | 10 | Cat | 368-383 |
| 10 | EAWFPEDQRVLTPN | 11 | Cat | 426-439 |
| 11 | ALPPSTHGAGWQLFCR | 12 | cat/crd | 443-458 |
| 12 | TVWSAHSGPTRMATAIAR | 13 | Crd | 459-476 |
| 13 | CSSFSRSGKRRGERM | 14 | Crd | 486-500 |
| 14 | HVLTGCSSHWEVEDLGT | 15 | Crd | 557-573 |
| 15 | PVLRPRGQPNQCVG | 16 | Crd | 577-590 |
| 16 | SALPGTSHVL | 17 | Crd | 636-645 |
| 17 | RDVSTTGSTSEEAVTAVAI | 18 | Crd | 659-677 |

pro = prodomain, cat = catalytic domain, crd = cysteine-rich domain

FIG. 2 is a depiction of a three-dimensional structural model of hPCSK9. The following sets of linear epitopes are proximal in the three-dimensional model: region 1 in the prodomain (SEQ ID NO 2 and SEQ ID NO 3); region 2 in the catalytic domain and the catalytic/cysteine-rich domain (SEQ ID NO 4 and SEQ ID NO 12); region 3 in the catalytic domain (SEQ ID NO 5, SEQ ID NO 7 and SEQ ID NO 10); region 4 in the catalytic domain (SEQ ID NO 6 and SEQ ID NO 8); region 5 in the catalytic domain (SEQ ID NO 9 and SEQ ID NO 11); region 6 in the cysteine-rich domain (SEQ ID NO13, SEQ ID NO 14 and SEQ ID NO 15); and region 7 in the cysteine-rich domain (SEQ ID NO 16, SEQ ID NO 17 and SEQ ID NO 18).

Amino acid residues in these sets of epitopes form non-linear epitopes.

The term “antibody” as used herein refers to an intact antibody or an antigen binding fragment (i.e., “antigen-binding portion”) or single chain (i.e., light or heavy chain) thereof. An intact antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding portion” of an antibody, as used herein, refers to one or more fragments of an intact antibody that retain the ability to specifically bind to a given antigen (e.g., hPCSK9). Antigen binding functions of an antibody can be performed by fragments of an intact antibody. Examples of binding fragments encompassed within the term “antigen binding portion” of an antibody include a Fab fragment, a monovalent fragment consisting of the V_{L}, V_{H}, C_{L} and CH1 domains; an F(ab)₂ fragment, a bivalent fragment comprising two Fab fragments (generally one from a heavy chain and one from a light chain) linked by a disulfide bridge at the hinge region; an Fd fragment consisting of the V_{H} and CH1 domains; an Fv fragment consisting of the V_{L} and V_{H} domains of a single arm of an antibody; a single domain antibody (dAb) fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by an artificial peptide linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies.

Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding portions can be incorporated into single chain molecules comprising a pair of tandem Fv segments (V_{H}-CH1-V_{H}-CH1) which, together with complementary light chain polypeptides, form a pair of antigen binding regions (Zapata et al., 1995 Protein Eng. 8(10):1057-1062; and U.S. Pat. No. 5,641,870).

An “isolated PCSK9 binding molecule”, as used herein, refers to a binding molecule that is substantially free of molecules having antigenic specificities for antigens other than PCSK9 (e.g., an isolated antibody that specifically binds hPCSK9 is substantially free of antibodies that specifically bind antigens other than hPCSK9). An isolated binding molecule that specifically binds hPCSK9 may, however, have cross-reactivity to other antigens, such as PCSK9 molecules from other species. A binding molecule is “purified” if it is substantially free of cellular material.

The term “monoclonal antibody composition” as used herein refers to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to an antibody displaying a single binding specificity that has variable regions in which both the framework and CDR regions are derived from human sequences. In one embodiment, the human monoclonal antibody is produced by a hybridoma that includes a B cell obtained from a transgenic nonhuman animal (e.g., a transgenic mouse having a genome comprising a human heavy chain transgene and a light chain transgene) fused to an immortalized cell.

The term “recombinant human antibody”, as used herein, includes any human antibody that is prepared, expressed, created or isolated by recombinant means, such as an antibody isolated from an animal (e.g., a mouse) that is transgenic or transchromosomal for human immunoglobulin genes or a hybridoma prepared therefrom; an antibody isolated from a host cell transformed to express the human antibody, e.g., from a transfectoma; an antibody isolated from a recombinant, combinatorial human antibody library; and an antibody prepared, expressed, created or isolated by any other means that involve splicing of all or a portion of a human immunoglobulin gene sequences to another DNA sequence. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in a human.

As used herein, “isotype” refers to the antibody class (e.g., IgM, IgE, IgG such as IgG1 or IgG4) that is encoded by the heavy chain constant region gene.

The phrases “an antibody recognizing an antigen” and “an antibody specific for an antigen” are used interchangeably herein with the term “an antibody that binds specifically to an antigen.”

As used herein, a PCSK9 binding molecule (e.g., an antibody or antigen binding portion thereof) that “specifically binds to PCSK9” is intended to refer to a PCSK9 binding molecule that binds to PCSK9 with a K_{D} of 1×10⁻⁷ M or less. A PCSK9 binding molecule (e.g., an antibody) that “cross-reacts with an antigen” is intended to refer to a PCSK9 binding molecule that binds that antigen with a K_{D} of 1×10⁻⁶ M or less. A PCSK9 binding molecule (e.g., an antibody) that “does not cross-react” with a given antigen is intended to refer to a PCSK9 binding molecule that either does not bind detectably to the given antigen, or binds with a K_{D} of 1×10⁻⁵ M or greater. In certain embodiments, such binding molecules that do not cross-react with the antigen exhibit essentially undetectable binding against these proteins in standard binding assays.

As used herein, the term “high affinity”, when referring to an IgG antibody, indicates that the antibody has a K_{D} of 10⁻⁹ M or less for a target antigen.

As used herein, the term “an epitope within or overlapping” particular amino acid residues refers to an epitope that comprises, consists of, or overlaps with all or a portion of such residues.

The term “epitope” or “antigenic determinant” refers to a site on an antigen to which a PCSK9 binding molecule of the invention specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Also encompassed by the present invention are PCSK9 binding molecules that bind to (i.e., recognize) the same epitope as the PCSK9 binding molecules described herein. PCSK9 binding molecules that bind to the same epitope can be identified by their ability to cross-compete with (i.e., competitively inhibit binding of) a reference PCSK9 binding molecule to a target antigen in a statistically significant manner. Competitive inhibition can occur, for example, if the PCSK9 binding molecules bind to identical or structurally similar epitopes (e.g., overlapping epitopes), or spatially proximal epitopes which, when bound, causes steric hindrance between the antibodies.

Competitive inhibition can be determined using routine assays in which the PCSK9 binding molecule under test inhibits specific binding of a reference PCSK9 binding molecule to a common antigen. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test PCSK9 binding molecule and a labeled reference PCSK9 binding molecule. Competitive inhibition is measured by determining the amount of label bound to the solid surface or cells in the presence of the test PCSK9 binding molecule. Usually the test PCSK9 binding molecule is present in excess. Usually, when a competing PCSK9 binding molecule is present in excess, it will inhibit specific binding of a reference PCSK9 binding molecule to a common antigen by at least 50-55%, 55-60%, 60-65%, 65-70% 70-75% or more.

Other techniques include, for example, epitope mapping methods, such as, x-ray analyses of crystals of antigen: PCSK9 binding molecule complexes which provides atomic resolution of the epitope. Other methods monitor the binding of the PCSK9 binding molecule to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the PCSK9 binding molecule of interest to affinity isolate specific short peptides from combinatorial phage display peptide libraries. The peptides are then regarded as leads for the definition of the epitope corresponding to the PCSK9 binding molecule used to screen the peptide library. For epitope mapping, computational algorithms have also been developed which have been shown to map conformational discontinuous epitopes.

As used herein, the term “subject” includes any human or nonhuman animal.

The term “nonhuman animal” includes all nonhuman vertebrates, e.g., mammals and non-mammals, such as nonhuman primates, rodents, rabbits, sheep, dogs, cats, horses, cows, birds, amphibians, reptiles, etc.

A nucleotide sequence is said to be “optimized” if it has been altered to encode an amino acid sequence using codons that are preferred in the production cell or organism, generally a eukaryotic cell, for example, a cell of a yeast such as Pichia, an insect cell, a mammalian cell such as Chinese Hamster Ovary cell (CHO) or a human cell. The optimized nucleotide sequence is engineered to encode an amino acid sequence identical or nearly identical to the amino acid sequence encoded by the original starting nucleotide sequence, which is also known as the “parental” sequence.

As used herein, the term “humaneered antibodies” means antibodies that bind the same epitope but differ in sequence. Example technologies include humaneered antibodies produced by humaneering technology of Kalobios, wherein the sequence of the antigen-hinging region is derived by, e.g., mutation, rather than due to conservative amino acid replacements (See e.g., WO2004/072266, WO2005/069970).

Various aspects of the invention are described in further detail in the following subsections.

Standard assays to evaluate the ability of molecules to bind to PCSK9 of various species, and particular epitopes of PCSK9, are known in the art, including, for example, ELISAs and western blots. Determination of whether a PCSK9 binding molecule binds to a specific epitope of PCSK9 can employ a peptide epitope competition assay. For example, a PCSK9 binding molecule is incubated with a peptide corresponding to a PCSK9 epitope of interest at saturating concentrations of peptide. The preincubated PCSK9 binding molecule is tested for binding to immobilized PCSK9, e.g., by Biacore® analysis. Inhibition of PCSK9 binding by preincubation with the peptide indicates that the PCSK9 binding molecule binds to the peptide epitope (see, e.g., U.S. Pat. Pub. 20070072797). Binding kinetics also can be assessed by standard assays known in the art, such as by Biacore® analysis. Assays to evaluate the effects of PCSK9 binding molecules on functional properties of PCSK9 are described in further detail below.

Accordingly, a PCSK9 binding molecule that “inhibits” one or more of these PCSK9 functional properties (e.g., biochemical, cellular, physiological or other biological activities, or the like), as determined according to methodologies known to the art and described herein, will be understood to produce a statistically significant decrease in the particular functional property relative to that seen in the absence of the binding molecule (e.g., when a control molecule of irrelevant specificity is present). A PCSK9 binding molecule that inhibits PCSK9 activity effects such a statistically significant decrease by at least 5% of the measured parameter. In certain embodiments, an antibody or other PCSK9 binding molecule may produce a decrease in the selected functional property of at least 10%, 20%, 30%, or 50% compared to control. In some embodiments, PCSK9 inhibition is determined by measuring LDL-R levels. An increase in LDL-R levels in the presence of a PCSK9 binding molecule indicates that the PCSK9 binding molecule inhibits PCSK9.

Antibodies

The anti-PCSK9 antibodies described herein include human monoclonal antibodies. In some embodiments, antigen binding portions of antibodies that bind to PCSK9, (e.g., V_{H} and V_{L} chains) are “mixed and matched” to create other anti-PCSK9 binding molecules. The binding of such “mixed and matched” antibodies can be tested using the aforementioned binding assays (e.g., ELISAs). When selecting a V_{H} to mix and match with a particular V_{L} sequence, typically one selects a V_{H} that is structurally similar to the V_{H} it replaces in the pairing with that V_{L}. Likewise a full length heavy chain sequence from a particular full length heavy chain/full length light chain pairing is generally replaced with a structurally similar full length heavy chain sequence. Likewise, a V_{L} sequence from a particular V_{H}/V_{L} pairing should be replaced with a structurally similar V_{L} sequence. Likewise a full length light chain sequence from a particular full length heavy chain/full length light chain pairing should be replaced with a structurally similar full length light chain sequence. Identifying structural similarity in this context is a process well known in the art.

In other aspects, the invention provides antibodies that comprise the heavy chain and light chain CDR1s, CDR2s and CDR3s of one or more PCSK9-binding antibodies, in various combinations. Given that each of these antibodies can bind to PCSK9 and that antigen-binding specificity is provided primarily by the CDR1, 2 and 3 regions, the V_{H} CDR1, 2 and 3 sequences and V_{L} CDR1, 2 and 3 sequences can be “mixed and matched” (i.e., CDRs from different antibodies can be mixed and matched). PCSK9 binding of such “mixed and matched” antibodies can be tested using the binding assays described herein (e.g., ELISAs). When V_{H} CDR sequences are mixed and matched, the CDR1, CDR2 and/or CDR3 sequence from a particular V_{H} sequence should be replaced with a structurally similar CDR sequence(s). Likewise, when V_{L} CDR sequences are mixed and matched, the CDR1, CDR2 and/or CDR3 sequence from a particular V_{L} sequence should be replaced with a structurally similar CDR sequence(s). Identifying structural similarity in this context is a process well known in the art.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes as the source of the sequences. In one such system, a human antibody is raised in a transgenic mouse carrying human immunoglobulin genes. The transgenic is immunized with the antigen of interest (e.g., an epitope of hPCSK9 described herein). Alternatively, a human antibody is identified by providing a human immunoglobulin gene library displayed on phage and screening the library with the antigen of interest (e.g., hPCSK9 or an hPCSK9 epitope described herein).

A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline-encoded sequence, due to, for example, naturally occurring somatic mutations or artificial site-directed mutations. However, a selected human antibody typically has an amino acid sequence at least 90% identical to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

The percent identity between two sequences is a function of the number of identity positions shared by the sequences (i.e., % identity=# of identity positions/total # of positions×100), taking into account the number of gaps, and the length of each gap, that need to be introduced for optimal alignment of the two sequences. The comparison of sequences and determination of percent identity between two sequences is determined using the algorithm of E. Meyers and W. Miller (1988 Comput. Appl. Biosci., 4:11-17) which has been incorporated into the ALIGN program (version 2.0), using a PAM120 weight residue table, a gap length penalty of 12 and a gap penalty of 4.

Typically, a V_{H} or V_{L} of a human antibody derived from a particular human germline sequence will display no more than 10 amino acid differences from the amino acid sequence encoded by the human germline immunoglobulin gene. In certain cases, the V_{H} or V_{L} of the human antibody may display no more than 5, or even no more than 4, 3, 2, or 1 amino acid difference from the amino acid sequence encoded by the germline immunoglobulin gene.

Camelid Antibodies

Antibody proteins obtained from members of the camel and dromedary (Camelus bactrianus and Calelus dromaderius) family, including New World members such as llama species (Lama paccos, Lama glama and Lama vicugna), have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See WO 94/04678.

A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies' being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier. See U.S. Pat. Pub. No. 20040161738, published Aug. 19, 2004. These features combined with the low antigenicity in humans indicate great therapeutic potential. Further, these molecules can be fully expressed in prokaryotic cells such as E. coli.

Accordingly, a feature of the present invention is a camelid antibody or camelid nanobody having high affinity for PCSK9. In certain embodiments herein, the camelid antibody or nanobody is naturally produced in the camelid animal, i.e., is produced by the camelid following immunization with PCSK9 or a peptide fragment thereof, using techniques described herein for other antibodies. Alternatively, the anti-PCSK9 camelid nanobody is engineered, i.e., produced by selection, for example from a library of phage displaying appropriately mutagenized camelid nanobody proteins using panning procedures with PCSK9 or a PCSK9 epitope described herein as a target. Engineered nanobodies can further be customized by genetic engineering to increase the half life in a recipient subject from 45 minutes to two weeks.

Diabodies

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{HA}-V_{LB} and V_{HB}-V_{LA} (V_{H}-V_{L} configuration), or V_{LA}-V_{HB} and V_{LB}-V_{HA} (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21).

A diabody can be fused to Fc to generate a “di-diabody” (see Lu et al., 2004 J. Biol. Chem., 279(4):2856-65).

Engineered and Modified Antibodies

An antibody of the invention can be prepared using an antibody having one or more V_{H} and/or V_{L} sequences as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998 Nature 332:323-327; Jones et al., 1986 Nature 321:522-525; Queen et al., 1989 Proc. Natl. Acad. See. U.S.A. 86:10029-10033; U.S. Pat. No. 5,225,539, and U.S. Pat. Nos. 5,530,101; 5,585,089; 5,693,762 and 6,180,370).

Framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., 1991 Sequences of Proteins of Immunological Interest, Fifth Edition, U.S. Department of Health and Human Services, NIH Publication No. 91-3242; Tomlinson et al., 1992 J. Mol. Biol. 227:776-798; and Cox et al., 1994 Eur. J. Immunol. 24:827-836; the contents of each of which are expressly incorporated herein by reference.

The V_{H} CDR1, 2 and 3 sequences and the V_{L} CDR1, 2 and 3 sequences can be grafted onto framework regions that have the identical sequence as that found in the germline immunoglobulin gene from which the framework sequence is derived, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

CDRs can also be grafted into framework regions of polypeptides other than immunoglobulin domains. Appropriate scaffolds form a conformationally stable framework that displays the grafted residues such that they form a localized surface and bind the target of interest (e.g., PCSK9). For example, CDRs can be grafted onto a scaffold in which the framework regions are based on fibronectin, ankyrin, lipocalin, neocarzinostain, cytochrome b, CP1 zinc finger, PST1, coiled coil, LACI-D1, Z domain or tendramisat (See e.g., Nygren and Uhlen, 1997 Current Opinion in Structural Biology, 7, 463-469).

Another type of variable region modification is mutation of amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s), and the effect on antibody binding, or other functional property of interest, can be evaluated in in vitro or in vivo assays as described herein. Conservative modifications can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Pat. Pub. No. 20030153043 by Can et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, U.S. Pat. No. 6,277,375 describes the following mutations in an IgG that increase its half-life in vivo: T252L, T254S, T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO 00/42072 by Presta. Moreover, the binding sites on human IgG1 for FcγR1, FcγRII, FcγRIII and FcRn have been mapped and variants with improved binding have been described (see Shields, R. L. et al., 2001 J. Biol. Chem. 276:6591-6604).

In still another embodiment, the glycosylation of an antibody is modified. For example, an aglycoslated antibody can be made (i.e., the antibody lacks glycosylation). Glycosylation can be altered, for example, to increase the affinity of the antibody for an antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Pub. WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO 99/54342 by Umana et al. describes cell lines engineered to express glycoprotein-modifying glycosyl transferases (e.g., beta(1,4)—N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG moieties become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

In addition, pegylation can be achieved in any part of a PCSK9 binding polypeptide of the invention by the introduction of a normatural amino acid. Certain normatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the normatural amino acid of choice. Particular normatural amino acids that are beneficial for purpose of conjugating moieties to the polypeptides of the invention include those with acetylene and azido side chains. The polypeptides containing these novel amino acids can then be pegylated at these chosen sites in the protein.

Methods of Engineering Antibodies

As discussed above, anti-PCSK9 antibodies can be used to create new anti-PCSK9 antibodies by modifying full length heavy chain and/or light chain sequences, V_{H} and/or V_{L} sequences, or the constant region(s) attached thereto. For example, one or more CDR regions of the antibodies can be combined recombinantly with known framework regions and/or other CDRs to create new, recombinantly-engineered, anti-PCSK9 antibodies. Other types of modifications include those described in the previous section. The starting material for the engineering method is one or more of the V_{H} and/or V_{L} sequences, or one or more CDR regions thereof. To create the engineered antibody, it is not necessary to actually prepare (i.e., express as a protein) an antibody having one or more of the V_{H} and/or V_{L} sequences, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Standard molecular biology techniques can be used to prepare and express the altered antibody sequence. The antibody encoded by the altered antibody sequence(s) is one that retains one, some or all of the functional properties of the anti-PCSK9 antibody from which it is derived, which functional properties include, but are not limited to, specifically binding to PCSK9, inhibiting autocatalytic cleavage, inhibiting LDL-R binding, inhibiting LDL-R degradation. The functional properties of the altered antibodies can be assessed using standard assays available in the art and/or described herein (e.g., ELISAs).

In certain embodiments of the methods of engineering antibodies of the invention, mutations can be introduced randomly or selectively along all or part of an anti-PCSK9 antibody coding sequence and the resulting modified anti-PCSK9 antibodies can be screened for binding activity and/or other functional properties (e.g., inhibiting autocatalytic cleavage, inhibiting LDL-R binding, inhibiting LDL-R degradation) as described herein. Mutational methods have been described in the art. For example, PCT Pub. WO 02/092780 by Short describes methods for creating and screening antibody mutations using saturation mutagenesis, synthetic ligation assembly, or a combination thereof. Alternatively, WO 03/074679 by Lazar et al. describes methods of using computational screening methods to optimize physiochemical properties of antibodies.

Non-Antibody PCSK9 Binding Molecules

The invention further provides PCSK9 binding molecules that exhibit functional properties of antibodies but derive their framework and antigen binding portions from other polypeptides (e.g., polypeptides other than those encoded by antibody genes or generated by the recombination of antibody genes in vivo). The antigen binding domains (e.g., PCSK9 binding domains) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain is a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest (e.g., hPCSK9) and for other functions (e.g., inhibition of biological activity of PCSK9). Selected clones can be used as the basis for further randomization and selection to produce derivatives of higher affinity for the antigen.

High affinity binding molecules are generated, for example, using the tenth module of fibronectin III (¹⁰Fn3) as the scaffold. A library is constructed for each of three CDR-like loops of ¹⁰FN3 at residues 23-29, 52-55, and 78-87. To construct each library, DNA segments encoding sequence overlapping each CDR-like region are randomized by oligonucleotide synthesis. Techniques for producing selectable ¹⁰Fn3 libraries are described in U.S. Pat. Nos. 6,818,418 and 7,115,396; Roberts and Szostak, 1997 Proc. Natl. Acad. Sci. USA 94:12297; U.S. Pat. No. 6,261,804; U.S. Pat. No. 6,258,558; and Szostak et al. WO98/31700.

Non-antibody binding molecules can be produces as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396.

Nucleic Acid Molecules Encoding Antibodies of the Invention

Another aspect of the invention pertains to nucleic acid molecules that encode the PCSK9 binding molecules of the invention. The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. A nucleic acid of the invention can be, for example, DNA or RNA and may or may not contain intronic sequences. In an embodiment, the nucleic acid is a cDNA molecule. The nucleic acid may be present in a vector such as a phage display vector, or in a recombinant plasmid vector.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from various phage clones that are members of the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as to a Fab light chain gene) by operatively linking the V_{L}-encoding DNA to another DNA molecule encoding the light chain constant region, CL. The sequences of human light chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or a lambda constant region.

To create an scFv gene, the V_{H}- and V_{L}-encoding DNA fragments are operatively linked to another fragment encoding a flexible linker, e.g., encoding the amino acid sequence (Gly4-Ser)₃, such that the V_{H} and V_{L} sequences can be expressed as a contiguous single-chain protein, with the V_{L} and V_{H} regions joined by the flexible linker (see e.g., Bird et al., 1988 Science 242:423-426; Huston et al., 1988 Proc. Natl. Acad. Sci. USA 85:5879-5883; McCafferty et al., 1990 Nature 348:552-554).

Monoclonal Antibody Generation

Monoclonal antibodies (mAbs) can be produced by a variety of techniques, including conventional monoclonal antibody methodology e.g., the standard somatic cell hybridization technique of Kohler and Milstein (1975 Nature, 256:495), or using library display methods, such as phage display.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539, and U.S. Pat. Nos. 5,530,101; 5,585,089; 5,693,762 and 6,180,370.

In a certain embodiment, the antibodies of the invention are human monoclonal antibodies. Such human monoclonal antibodies directed against PCSK9 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see, e.g., Lonberg et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N.Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Pub. Nos. WO 92103918, WO 93/12227, WO 94/25585, WO 97113852, WO 98/24884 and WO 99/45962, all to Lonberg and Kay; and PCT Pub. No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, are described in detail in WO 02/43478.

Still further, alternative transgenic animal systems expressing human immunoglobulin genes are available in the art and can be used to raise anti-PCSK9 antibodies of the invention. For example, an alternative transgenic system referred to as the Xenomouse® (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-PCSK9 antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise anti-PCSK9 antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Libraries can be screened for binding to full length PCSK9 or to a particular epitope of PCSK9.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Generation of Human Monoclonal Antibodies in Human Ig Mice

Purified recombinant human PCSK9 expressed in prokaryotic cells (e.g., E. coli) or eukaryotic cells (e.g., mammalian cells, e.g., HEK293 cells) can be used as the antigen. The protein can be conjugated to a carrier, such as keyhole limpet hemocyanin (KLH).

Fully human monoclonal antibodies to PCSK9 are prepared using HCo7, HCo12 and HCo17 strains of HuMab transgenic mice and the KM strain of transgenic transchromosomic mice, each of which express human antibody genes. In each of these mouse strains, the endogenous mouse kappa light chain gene can be homozygously disrupted as described in Chen et al., 1993 EMBO J. 12:811-820 and the endogenous mouse heavy chain gene can be homozygously disrupted as described in Example 1 of WO 01109187. Each of these mouse strains carries a human kappa light chain transgene, KCo5, as described in Fishwild et al., 1996 Nature Biotechnology 14:845-851. The HCo7 strain carries the HCo7 human heavy chain transgene as described in U.S. Pat. Nos. 5,545,806; 5,625,825; and 5,545,807. The HCo12 strain carries the HCo12 human heavy chain transgene as described in Example 2 of WO 01/09187. The HCo17 stain carries the HCo17 human heavy chain transgene. The KNM strain contains the SC20 transchromosome as described in WO 02/43478.

To generate fully human monoclonal antibodies to PCSK9, HuMab mice and KM mice are immunized with purified recombinant PCSK9, a PCSK9 fragment, or a conjugate thereof (e.g., PCSK9-KLH) as antigen. General immunization schemes for HuMab mice are described in Lonberg, N. et al., 1994 Nature 368(6474): 856-859; Fishwild, D. et al., 1996 Nature Biotechnology 14:845-851 and WO 98/24884. The mice are 6-16 weeks of age upon the first infusion of antigen. A purified recombinant preparation (5-50 μg) of the antigen is used to immunize the HuMab mice and KM mice in the peritoneal cavity, subcutaneously (Sc) or by footpad injection.

Transgenic mice are immunized twice with antigen in complete Freund's adjuvant or Ribi adjuvant either in the peritoneal cavity (IP), subcutaneously (Sc) or by footpad (FP), followed by 3-21 days IP, Sc or FP immunization (up to a total of 11 immunizations) with the antigen in incomplete Freund's or Ribi adjuvant. The immune response is monitored by retroorbital bleeds. The plasma is screened by ELISA, and mice with sufficient titers of anti-PCSK9 human immunogolobulin are used for fusions. Mice are boosted intravenously with antigen 3 and 2 days before sacrifice and removal of the spleen. Typically, 10-35 fusions for each antigen are performed. Several dozen mice are immunized for each antigen. A total of 82 mice of the HCo7, HCo12, HCo17 and KM mice strains are immunized with PCSK9.

To select HuMab or KM mice producing antibodies that bound PCSK9, sera from immunized mice can be tested by ELISA as described by Fishwild, D. et al., 1996. Briefly, microtiter plates are coated with purified recombinant PCSK9 at 1-2 μg/ml in PBS, 50 μl/wells incubated 4° C. overnight then blocked with 200 μl/well of 5% chicken serum in PBS/Tween (0.05%). Dilutions of plasma from PCSK9-immunized mice are added to each well and incubated for 1-2 hours at ambient temperature. The plates are washed with PBS/Tween and then incubated with a goat-anti-human IgG Fc polyclonal antibody conjugated with horseradish peroxidase (HRP) for 1 hour at room temperature. After washing, the plates are developed with ABTS substrate (Sigma, A-1888, 0.22 mg/ml) and analyzed by spectrophotometer at OD 415-495. Splenocytes of mice that developed the highest titers of anti-PCSK9 antibodies are used for fusions. Fusions are performed and hybridoma supernatants are tested for anti-PCSK9 activity by ELISA.

The mouse splenocytes, isolated from the HuMab mice and KM mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the number of SP2/0 nonsecreting mouse myeloma cells (ATCC, CRL 1581) with 50% PEG (Sigma). Cells are plated at approximately 1×10⁵/well in flat bottom microtiter plates, followed by about two weeks of incubation in selective medium containing 10% fetal bovine serum, 10% P388D 1 (ATCC, CRL TIB-63) conditioned medium, 3-5% Origen® (IGEN) in DMEM (Mediatech, CRL 10013, with high glucose, L-glutamine and sodium pyruvate) plus 5 mM HEPES, 0.055 mM 2-mercaptoethanol, 50 μg/ml gentamycin and 1×HAT (Sigma, CRL P-7185). After 1-2 weeks, cells are cultured in medium in which the HAT is replaced with HT. Individual wells are then screened by ELISA for human anti-PCSK9 monoclonal IgG antibodies. Once extensive hybridoma growth occurred, medium is monitored usually after 10-14 days. The antibody secreting hybridomas are replated, screened again and, if still positive for human IgG, anti-PCSK9 monoclonal antibodies are subcloned at least twice by limiting dilution. The stable subclones are then cultured in vitro to generate small amounts of antibody in tissue culture medium for further characterization.

Generation of Hybridomas Producing Human Monoclonal Antibodies

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Cells are plated at approximately 2×145 in flat bottom microtiter plates, followed by a two week incubation in selective medium containing 20% fetal Clone Serum, 18% “653” conditioned media, 5% Origen® (IGEN), 4 mM L-glutamine, 1 mM sodium pyruvate, 5 mM HEPES, 0:055 mM 2-mercaptoethanol, 50 units/ml penicillin, 50 μg/ml streptomycin, 50 μg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using an extinction coefficient of 1.43. The monoclonal antibodies can be aliquoted and stored at −80° C.

Generation of Transfectomas Producing Monoclonal Antibodies

Antibodies of the invention also can be produced in a host cell transfectoma using, for example, a combination of recombinant DNA techniques and gene transfection methods as is well known in the art (e.g., Morrison, 1985 Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. 1990 Methods in Enzymology 185, Academic Press, San Diego, Calif.). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SR^{a} promoter system, which contains sequences from the SV40 early promoter and the long terminal repeat of human T cell leukemia virus type 1 (Takebe et al., 1988 Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr− host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the invention in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood, 1985 Immunology Today 6:12-13).

Mammalian host cells for expressing the recombinant antibodies of the invention include Chinese Hamster Ovary (CHO cells) (including dhfr− CHO cells, described Urlaub and Chasin, 1980 Proc. Natl. Acad. Sci. USA 77:4216-4220 used with a DH FR selectable marker, e.g., as described in Kaufman and Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

Bispecific Molecules

In another aspect, the present invention features bispecific molecules comprising a PCSK9 binding molecule (e.g., an anti-PCSK9 antibody, or a fragment thereof), of the invention. A PCSK9 binding molecule of the invention can be derivatized or linked to another functional molecule, e.g., another peptide or protein (e.g., another antibody or ligand for a receptor) to generate a bispecific molecule that binds to at least two different binding sites or target molecules. The PCSK9 binding molecule of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for PCSK9 and a second binding specificity for a second target epitope.

In one embodiment, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

The bispecific molecules of the present invention can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al., 1984 J. Exp. Med. 160:1686; Liu et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand x Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

Functional Assays

The functional characteristics of PCSK9 binding molecules can be tested in vitro and in vivo. For example, binding molecules can be tested for the ability to inhibit interaction of PCSK9 with LDL-R, inhibition of PCSK9-dependent effects on LDL-R (e.g., LDL-R mediated uptake of LDL-c), inhibition of PCSK9 proteolytic activity, and decrease LDL-c in vivo.

PCSK9 binding to LDL-R can be detected using Biacore® by immobilizing LDL-R to a solid support and detecting soluble PCSK9 binding to the LDL-R. Alternatively, PCSK9 can be immobilized, and LDL-R binding can be detected. PCSK-9/LDL-R binding can also be analyzed by ELISA (e.g., by detecting PCSK9 binding to immobilized LDL-R), or by fluorescence resonance energy transfer (FRET). To perform FRET, fluorophore-labeled PCSK9 binding to LDL-R in solution can be detected (see, for example, U.S. Pat. No. 5,631,169).

PCSK9 binding to LDL-R has been detected by coimmunoprecipitation (Lagace et al., 2006 J. Clin. Inv. 116(11):2995-3005). To examine PCSK9-LDL-R binding in this manner, HepG2 cells are cultured in sterol-depleted medium for 18 hours. Purified PCSK9 is added to the medium in the presence of 0.1 mM chloroquine and the cells are incubated for one hour. Cells are lysed in mild detergent (1% digitonin w/vol). PCSK9 or LDL-R is immunoprecipitated from cell lysates, separated by SDS-PAGE, and immunoblotted to detect the presence of coimmunoprecipitated LDL-R or PCSK9, respectively (Lagace et al., 2006 J. Clin. Inv. 116(11):2995-3005). These assays may be conducted with a mutant form of PCSK9 that binds to LDL-R with a higher avidity (e.g., hPCSK9 D374Y) (Lagace et al., 2006, supra).

Hepatocytes express LDL-R on the cell surface. Addition of purified PCSK9 to cultured hepatocyte cells (e.g., HepG2 cells, ATCC, HB-8065) produces a decrease in LDL-R expression in a dose- and time-dependent manner (Lagace et al., 2006 J. Clin. Inv. 116(11):2995-3005). PCSK9 binding molecules can be tested for the ability to increase LDL-R levels by hepatocytes. For example, HepG2 cells are cultured in sterol-depleted medium (DMEM supplemented with 100 U/ml penicillin, 100 μg/ml streptomycin sulfate, and 1 g/l glucose, 5% (vol/vol) newborn calf lipoprotein-deficient serum (NCLPDS), 10 μM sodium compactin, and 50 μM sodium mevalonate) for 18 hours to induce LDL-R expression. Purified PCSK9 (5 μg/ml) is added to the medium. LDL-R levels in cells harvested at 0, 0.5, 1, 2, and 4 hours after addition of PCSK9 is determined (Lagace et al., 2006 J. Clin. Inv. 116(11):2995-3005). LDL-R levels can be determined by flow cytometry, FRET, immunoblotting, or other means.

LDL-c uptake by cells (e.g., HepG2 cells) can be measured using fluorescently-labeled LDL-c, DiI-LDL (3,3′-dioctadecylindocarbocyanine-low density lipoprotein) as described by Stephan and Yurachek (1993 J. Lipid Res. 34:325-330). Briefly, cells are incubated in culture with DiI-LDL (20-100 μg protein/ml) at 37° C. for 2 hours. Cells are washed, lysed, and the concentration of internalized DiI-LDL is quantitated using a spectrofluorometer. LDL-c uptake can be measured in cells contacted with a PCSK9 binding agent (prior to, and/or during the period in which DiI-LDL is present in the cell culture).

PCSK9 proteolytic activity can be measured in vitro using synthetic peptide substrates. See, e.g, Seidah et al., 2003 Proc. Natl. Acad. Sci. USA, 100(3):928-933. In one exemplary method, purified PCSK9 is incubated at 37° C. for 3-18 hours with 50 μM Suc-RPFHLLVY-MCA (4-methylcoumarin-7-amide) in 25 mM Tris/Mes, pH 7.4+2.5 mM CaCl₂ and 0.5% SDS. Fluorescence and matrix-assisted laser desorption ionization time-of-flight analysis of the products is used to detect cleavage products (Seidah et al., 2003 Proc. Natl. Acad. Sci. USA, 100(3):928-933; Basak et al., 2002 FEBS Lett. 514:333-339). This assay may be used to detect differences in cleavage efficiency in the presence of PCSK9 binding molecules.

Transgenic mice overexpressing human PCSK9 in liver have increased levels of plasma LDL-c relative to non-transgenic mice (Lagace et al., 2006 J. Clin. Inv. 116(11):2995-3005). See also Maxwell and Breslow, 2004 Proc. Natl. Acad. Sci. USA, 101:7100, describing overexpression of PCSK9 using an adenovirus vector in mice. PCSK9^{-/-} mice have been produced (Rashid et al., 2005 Proc. Natl. Acad. Sci. 102(5):5374-5379). These mice can be genetically modified to express a hPCSK9 transgene. PCSK9 binding molecules can be tested in any of these models, or in animals which are not genetically modified, for the ability to clear or reduce total cholesterol and/or LDL-c.

The kinetics of LDL clearance from plasma can be determined by injecting animals with [¹²⁵I]-labelled LDL, obtaining blood samples at 0, 5, 10, 15, and 30 minutes after injection, and quantitating [¹²⁵I]-LDL in the samples (Rashid et al., 2005 Proc. Natl. Acad. Sci. 102(5):5374-5379). The rate of LDL clearance is increased in PCSK9^{-/-} mice relative to wild type mice (Rashid et al., 2005 supra). Increased LDL clearance in animals administered a PCSK9 binding molecule indicates that the agent inhibits PCSK9 activity in vivo.

Decreases in total plasma cholesterol, plasma triglycerides, or LDL-c in response to treatment with a PCSK9 binding molecule are indicative of therapeutic efficacy of the PCKS9 binding molecule. Cholesterol and lipid profiles can be determined by colorimetric, gas-liquid chromatographic, or enzymatic means using commercially available kits.

Pharmaceutical Compositions

In another aspect, the present invention provides a composition, e.g., a pharmaceutical composition, containing one or a combination of PCSK9 binding molecules (e.g., monoclonal antibodies, or antigen-binding portion(s) thereof), of the present invention, formulated together with a pharmaceutically acceptable carrier. Such compositions may include one or a combination of (e.g., two or more different) binding molecules. For example, a pharmaceutical composition of the invention can comprise a combination of antibodies or agents that bind to different epitopes on the target antigen or that have complementary activities.

Pharmaceutical compositions of the invention also can be administered in combination therapy, i.e., combined with other agents. For example, the combination therapy can include an anti-PCSK9 antibody combined with at least one other cholesterol-reducing agent. Examples of therapeutic agents that can be used in combination therapy are described in greater detail below in the section on uses of the agents of the invention.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Dosage regimens for PCSK9 binding molecule of the invention include 1 mg/kg body weight or 3 mg/kg body weight by intravenous administration, with the antibody being given using one of the following dosing schedules: every four weeks for six dosages, then every three months; every three weeks; 3 mg/kg body weight once followed by 1 mg/kg body weight every three weeks.

In some methods, two or more binding molecules (e.g., monoclonal antibodies) with different binding specificities are administered simultaneously, in which case the dosage of each antibody administered falls within the ranges indicated. The PCSK9 binding molecule is usually administered on multiple occasions. Intervals between single dosages can be, for example, weekly, monthly, every three months or yearly. Intervals can also be irregular as indicated by measuring blood levels of binding molecule to PCSK9 in the patient. In some methods, dosage is adjusted to achieve a plasma concentration of the PCSK9 binding molecule of about 1-1000 μg/ml and in some methods about 25-300 μg/ml.

Alternatively, a PCSK9 binding molecule can be administered as a sustained release formulation, in which case less frequent administration is required. Dosage and frequency vary depending on the half-life of the PCSK9 binding molecule in the patient. In general, human antibodies show the longest half-life, followed by humanized antibodies, humaneered antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of PCSK9 binding molecule of the invention can results in a decrease in severity of disease symptoms (e.g., a decrease in plasma cholesterol, or a decrease in a symptom of a cholesterol-related disorder), an increase in frequency and duration of disease symptom-free periods, or a prevention of impairment or disability due to the disease affliction.

A composition of the present invention can be administered by one or more routes of administration using one or more of a variety of methods known in the art. As will be appreciated by the skilled artisan, the route and/or mode of administration will vary depending upon the desired results. Routes of administration for PCSK9 binding molecules of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a PCSK9 binding molecule of the invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. Pat. No. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

In certain embodiments, the PCSK9 binding molecules of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chemother. 39:180); surfactant protein A receptor (Briscoe et al., 1995 Am. J. Physiol. 1233:134); p120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

Uses and Methods of the Invention

The PCSK9 binding molecules described herein have in vitro and in vivo diagnostic and therapeutic utilities. For example, these molecules can be administered to cells in culture, e.g. in vitro or in vivo, or in a subject, e.g., in vivo, to treat, prevent or diagnose a variety of disorders. PCSK9 binding molecules are particularly suitable for treating human patients having, or at risk for, elevated cholesterol or a condition associated with elevated cholesterol, e.g., a lipid disorder (e.g., hyperlipidemia, type I, type II, type III, type IV, or type V hyperlipidemia, secondary hypertriglyceridemia, hypercholesterolemia, xanthomatosis, cholesterol acetyltransferase deficiency). PCSK9 binding molecules are also suitable for treating human patients having, ateriosclerotic conditions (e.g., atherosclerosis), coronary artery disease, cardiovascular disease, and patients at risk for these disorders, e.g., due to the presence of one or more risk factors (e.g., hypertension, cigarette smoking, diabetes, obesity, or hyperhomocysteinemia).

When PCSK9 binding molecules are administered together with another agent, the two can be administered sequentially in either order or simultaneously. In some embodiments, a PCSK9 binding molecule is administered to a subject who is also receiving therapy with a second agent (e.g., a second cholesterol-reducing agent). Cholesterol reducing agents include statins, bile acid sequestrants, niacin, fibric acid derivatives, and long chain alpha, omego-dicarboxylic acids. Statins inhibit cholesterol synthesis by blocking HMGCoA, a key enzyme in cholesterol biosynthesis. Examples of statins are lovastatin, pravastatin, atorvastatin, cerivastatin, fluvastatin, and simvastatin. Bile acid sequestrants interrupt the recycling of bile acids from the intestine to the liver. Examples of these agents are cholestyramine and colestipol hydrochloride. Examples of fibric acid derivatives are clofibrate and gemfibrozil. Long chain alpha,omego-dicarboxylic acids are described, e.g., by Bisgaier et al., 1998, J. Lipid Res. 39:17-30; WO 98/30530; U.S. Pat. No. 4,689,344; WO 99/00116; U.S. Pat. No. 5,756,344; U.S. Pat. No. 3,773,946; U.S. Pat. No. 4,689,344; U.S. Pat. No. 4,689,344; U.S. Pat. No. 4,689,344; and U.S. Pat. No. 3,930,024); ethers (see, e.g., U.S. Pat. No. 4,711,896; U.S. Pat. No. 5,756,544; U.S. Pat. No. 6,506,799). Phosphates of dolichol (U.S. Pat. No. 4,613,593), and azolidinedione derivatives (U.S. Pat. No. 4,287,200) can also be used to reduce cholesterol levels.

A combination therapy regimen may be additive, or it may produce synergistic results (e.g., reductions in cholesterol greater than expected for the combined use of the two agents). In some embodiments, combination therapy with a PCSK9 binding molecule and a statin produces synergistic results (e.g., synergistic reductions in cholesterol). In some subjects, this can allow reduction in statin dosage to achieve the desired cholesterol levels.

PCSK9 binding molecules are useful for subjects who are intolerant to therapy with another cholesterol-reducing agent, or for whom therapy with another cholesterol-reducing agent has produced inadequate results (e.g., subjects who experience insufficient LDL-c reduction on statin therapy).

A PCSK9 binding molecule described herein can be administered to a subject with elevated cholesterol (e.g., a human subject with total plasma cholesterol levels of 200 mg/dl or greater, a human subject with LDL-c levels of 160 mg/dl or greater).

In one embodiment, the binding molecules of the invention can be used to detect levels of PCSK9. This can be achieved, for example, by contacting a sample (such as an in vitro sample) and a control sample with the PCSK9 binding molecule under conditions that allow for the formation of a complex between the binding molecule and PCSK9. Any complexes formed between the molecule and PCSK9 are detected and compared in the sample and the control. For example, standard detection methods, well known in the art, such as ELISA and flow cytometric assays, can be performed using the compositions of the invention.

Accordingly, in one aspect, the invention further provides methods for detecting the presence of PCSK9 (e.g., hPCSK9) in a sample, or measuring the amount of PCSK9, comprising contacting the sample, and a control sample, with a PCSK9 binding molecule (e.g., an antibody) of the invention, under conditions that allow for formation of a complex between the antibody or portion thereof and PCSK9. The formation of a complex is then detected, wherein a difference in complex formation between the sample compared to the control sample is indicative of the presence of PCSK9 in the sample.

Also within the scope of the invention are kits consisting of the compositions of the invention and instructions for use. The kit can further contain a least one additional reagent, or one or more additional antibodies of the invention (e.g., an antibody having a complementary activity which binds to an epitope on the target antigen distinct from the first antibody). Kits typically include a label indicating the intended use of the contents of the kit. The term label includes any writing, or recorded material supplied on or with the kit, or which otherwise accompanies the kit.

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

### EXAMPLES

### Example 1

### Generation of Human Antibodies by Phage Display

For the generation of antibodies against hPCSK9, selections with the MorphoSys HuCAL GOLD® phage display library are carried out. HuCAL GOLD® is a Fab library based on the HuCAL® concept in which all six CDRs are diversified, and which employs the CysDisplay™ technology for linking Fab fragments to the phage surface (Knappik et al., 2000 J. Mol. Biol. 296:57-86; Krebs et al., 2001 J Immunol. Methods 254:67-84; Rauchenberger et al., 2003 J Biol Chem. 278(40):38194-38205; WO 01/05950, Lohning, 2001).

Phagemid Rescue, Phage Amplification, and Purification

The HuCAL GOLD® library is amplified in 2×YT medium containing 34 μg/mlchloramphenicol and 1% glucose (2×YT-CG). After infection with hyperphage helper phages at an OD₆₀₀ₙₘ of 0.5 (30 mM at 37° C. without shaking; 30 min at 37° C. shaking at 250 rpm), cells are spun down (4120 g; 5 min; 4° C.), resuspended in 2×YT/34 μg/mlchloramphenicol/50 μg/ml kanamycin/0.25 mM IPTG and grown overnight at 22° C. Phages are PEG-precipitated twice from the supernatant, resuspended in PBS/20% glycerol and stored at −80° C.

Phage amplification between two panning rounds is conducted as follows: mid-log phase E. coli TG1 cells are infected with eluted phages and plated onto LB-agar supplemented with 1% of glucose and 34 μg/ml of chloramphenicol (LB-CG plates). After overnight incubation at 30° C., the TG1 colonies are scraped off the agar plates and used to inoculate 2×YT-CG until an OD₆₀₀ₙₘ of 0.5 is reached and hyperphage helper phages added for infection as described above.

Pannings with HuCAL GOLD®

For the selection of antibodies recognizing hPCSK9 two different panning strategies were applied. In summary, HuCAL GOLD® phage-antibodies are divided into four pools comprising different combinations of VH master genes (pool 1: VH1/5λκ, pool 2: VH3λκ, pool 3: VH2/4/6λκ, pool 4: VH1-6λκ). These pools are individually subjected to three rounds of solid phase panning on human hPCSK9 directly coated to Maxisorp plates and in addition three of solution pannings on biotinylated hPCSK9.

The first panning variant is solid phase panning against hPCSK9: 2 wells on a Maxisorp plate (F96 Nunc-Immunoplate) are coated with 300 μl of 5 μg/ml PCSK9-each o/n at 4° C. The coated wells are washed 2× with 350 μl PBS and blocked with 350 μl 5% MPBS for 2 h at RT on a microtiter plate shaker. For each panning about 10¹³ HuCAL GOLD® phage-antibodies are blocked with equal volume of PBST/5% MP for 2 h at room temperature. The coated wells are washed 2× with 350 μl PBS after the blocking. 300 μl of pre-blocked HuCAL GOLD® phage-antibodies are added to each coated well and incubated for 2 h at RT on a shaker. Washing is performed by adding five times 350 μl PBS/0.05% Tween, followed by washing another four times with PBS. Elution of phage from the plate is performed with 300 μl 20 mM DTT in 10 mM Tris/HCl pH8 per well for 10 min. The DTT phage eluate is added to 14 ml of E. coli TG1, which are grown to an OD₆₀₀ of 0.6-0.8 at 37° C. in 2YT medium and incubated in 50 ml plastic tubes for 45 min at 37° C. without shaking for phage infection. After centrifugation for 10 min at 5000 rpm, the bacterial pellets are each resuspended in 500 μl 2×YT medium, plated on 2×YT-CG agar plates and incubated overnight at 30° C. Colonies are then scraped from the plates and phages were rescued and amplified as described above. The second and third rounds of the solid phase panning on directly coated hPCSK9 is performed according to the protocol of the first round, but with increased stringency in the washing procedure.

The second panning variant is solution panning against biotinylated human hPCSK9: For the solution panning, using biotinylated hPCSK9 coupled to Dynabeads M-280 (Dynal), the following protocol is applied: 1.5 ml Eppendorf tubes are blocked with 1.5 ml 1% bovine serum albumin in PBS over night at 4° C. 200 μl streptavidin coated magnetic Dynabeads M-280 (Dynal) are washed 1× with 200 μl PBS and resuspended in 200 μl 1× Chemiblocker (diluted in 1×PBS). Blocking of beads is performed in pre-blocked tubes over night at 4° C. Phages diluted in 500 μl PBS for each panning condition are mixed with 500 μl 2× Chemiblocker/0.1% Tween 1 h at RT (rotator). Pre-adsorption of phages is performed twice: 50 μl of blocked Streptavidin magnetic beads are added to the blocked phages and incubated for 30 min at RT on a rotator. After separation of beads via a magnetic device (Dynal MPC-E) the phage supernatant (−1 ml) is transferred to a new blocked tube and pre-adsorption was repeated on 50 μl blocked beads for 30 min. Then, 200 nM biotinylated hPCSK9 is added to blocked phages in a new blocked 1.5 ml tube and incubated for 1 h at RT on a rotator. 100 μl of blocked streptavidin magnetic beads is added to each panning phage pool and incubated 10 min at RT on a rotator. Phages bound to biotinylated hPCSK9 are immobilized to the magnetic beads and collected with a magnetic particle separator (Dynal MPC-E). Beads are then washed 7× in PBS/0.05% Tween using a rotator, followed by washing another three times with PBS. Elution of phage from the Dynabeads is performed adding 300 μl 20 mM DTT in 10 mM Tris/HCl pH 8 to each tube for 10 min. Dynabeads are removed by the magnetic particle separator and the supernatant is added to 14 ml of an E. coli TG-1 culture grown to OD₆₀₀ₙₘ of 0.6-0.8. Beads are then washed once with 20 μl PBS and together with additionally removed phages the PBS was added to the 14 ml E. coli TG-1 culture. For phage infection, the culture is incubated in 50 ml plastic tubes for 45 min at 37° C. without shaking. After centrifugation for 10 min at 5000 rpm, the bacterial pellets are each resuspended in 500 μl 2×YT medium, plated on 2×YT-CG agar plates and incubated overnight at 30° C. Colonies are then scraped from the plates, and phages are rescued and amplified as described above.

The second and third rounds of the solution panning on biotinylated hPCSK9 are performed according to the protocol of the first round, except with increased stringency in the washing procedure.

Subcloning and Expression of Soluble Fab Fragments

The Fab-encoding inserts of the selected HuCAL GOLD® phagemids are sub-cloned into the expression vector pMORPH®X9_Fab_FH to facilitate rapid and efficient expression of soluble Fabs. For this purpose, the plasmid DNA of the selected clones is digested with XbaI and EcoRI, thereby excising the Fab-encoding insert (ompA-VLCL and phoA-Fd), and cloned into the XbaI/EcoRI-digested expression vector pMORPH®X9_Fab_FH. Fabs expressed from this vector carry two C-terminal tags (FLAG™ and 6×His, respectively) for both, detection and purification.

Microexpression of HuCAL GOLD® Fab Antibodies in E. coli

Chloramphenicol-resistant single colonies obtained after subcloning of the selected Fabs into the pMORPH®X9_Fab_FH expression vector are used to inoculate the wells of a sterile 96-well microtiter plate containing 100 μl 2×YT-CG medium per well and grown overnight at 37° C. 5 μl of each E. coli TG-1 culture is transferred to a fresh, sterile 96-well microtiter plate pre-filled with 100 μl 2×YT medium supplemented with 34 μg/ml chloramphenicol and 0.1% glucose per well. The microtiter plates are incubated at 30° C. shaking at 400 rpm on a microplate shaker until the cultures are slightly turbid (˜2-4 hrs) with an OD₆₀₀ₙₘ of ˜0.5.

To these expression plates, 20 μl 2×YT medium supplemented with 34 μg/ml chloramphenicol and 3 mM IPTG (isopropyl-β-D-thiogalactopyranoside) is added per well (end concentration 0.5 mM IPTG), the microtiter plates are sealed with a gas-permeable tape, and the plates are incubated overnight at 30° C. shaking at 400 rpm.

Generation of whole cell lysates (BEL extracts): Bacterial cells pellets were frozen on dry ice and then resuspended in PBS containing 1 mg/ml lysozyme, 2 mM MgCl₂ and benzonase and incubated for 1 hour on shaker. Lysates were blocked by the addition of 1% final concentration BSA and cleared lysates were added to appropriately coated ELISA plates to assess binding to PCSK9. The BEL extracts were used for binding analysis by ELISA.

Enzyme Linked Immunosorbent Assay (ELISA) Techniques

5 μg/ml of human recombinant hPCSK9 in PBS is coated onto 384 well Maxisorp plates (Nunc-Immunoplate) o/n at 4° C. After coating, the wells are washed once with PBS/0.05% Tween (PBS-T) and 2× with PBS. Then the wells are blocked with PBS-T with 2% BSA for 2 h at RT. In parallel, 15 μl BEL extract and 15 μl PBS-T with 2% BSA are incubated for 2 h at RT. The blocked Maxisorp plated are washed 3× with PBS-T before 10 μl of the blocked BEL extracts are added to the wells and incubated for 1 h at RT. For detection of the primary Fab antibodies, the following secondary antibodies are applied: alkaline phosphatase (AP)-conjugated AffiniPure F(ab′)₂ fragment, goat anti-human, -anti-mouse or -anti-sheep IgG (Jackson Immuno Research). For the detection of AP-conjugates fluorogenic substrates like AttoPhos (Roche) are used according to the instructions by the manufacturer. Between all incubation steps, the wells of the microtiter plate are washed with PBS-T three times and three times after the final incubation with secondary antibody. Fluorescence can be measured using Thermo Multiskan plate reader.

Expression of HuCAL GOLD® Fab Antibodies in E. coli and Purification

Expression of Fab fragments encoded by pMORPH⁶X9_Fab_FH in TG-1 cells is carried out in shaker flask cultures using 750 ml of 2×YT medium supplemented with 34 μg/ml chloramphenicol. Cultures are shaken at 30° C. until the OD₆₀₀ₙₘ reaches 0.5. Expression is induced by addition of 0.75 mM IPTG for 20 h at 30° C. Cells are disrupted using lysozyme and Fab fragments isolated by Ni-NTA chromatography (Qiagen, Hilden, Germany). Protein concentrations can be determined by UV-spectrophotometry (Krebs et al. J Immunol Methods 254, 67-84 (2001).

### Example 2

### Affinity Maturation of Selected Anti-PCSK9 Fabs by Parallel Exchange of LCDR3 and HCDR2 Cassettes

Generation of Fab Libraries for Affinity Maturation

In order to increase the affinity and inhibitory activity of the identified anti-PCSK9 antibodies, Fab clones are subjected to affinity maturation. For this purpose, CDR regions are optimized by cassette mutagenesis using trinucleotide directed mutagenesis (Virnekas et al. Nucleic Acids Res 22, 5600-5607, 1994).

The following paragraph briefly describes a protocol that can be used for cloning of the maturation libraries and Fab optimization. Fab fragments from expression vector pMORPH®X9_Fab_FH are cloned into the phagemid vector pMORPH®25 (U.S. Pat. No. 6,753,136). Two different strategies are applied in parallel to optimize both, the affinity and the efficacy of the parental Fabs.

Phage antibody Fab libraries are generated where the LCDR3 of six selected maturation candidates (“parental” clones) is replaced by a repertoire of individual light chain CDR3 sequences. In parallel, the HCDR2 region of each parental clone is replaced by a repertoire of individual heavy chain CDR2 sequences. Affinity maturation libraries are generated by standard cloning procedures and transformation of the diversified clones into electro-competent E. coli TOP10F′ cells (Invitrogen). Fab-presenting phages are prepared as described in Example 1. Maturation pools corresponding to each library are built and kept separate during the subsequent selection process.

Maturation Panning Strategies

Pannings using the four antibody pools are performed on biotinylated recombinant hPCSK9 in solution for three rounds, respectively as described in Example 1, solution panning against biotinylated hPCSK9. The selection stringency is increased by reduction of biotinylated antigen from panning round to panning round, by prolonged washing steps and by addition of non-biotinylated antigen for off-rate selection.

Electrochemiluminescene (BioVeris) Based Binding Analysis for Detection of hPCSK9 Binding Fab in Bacterial Lysates

Binding of optimized Fab antibodies in E. coli lysates (BEL extracts) to hPCSK9 is analyzed in BioVeris M-SERIES® 384 AnalyzerBioVeris, Europe, Witney, Oxforfshire, UK). BEL extracts are diluted in assay buffer (PBS/0.05% Tween20/0.5% BSA) for use in BioVeris screening. Biotinylated hPCSK9 is coupled to streptavidin coated paramagnetic beads, Anti-human (Fab)′₂ (Dianova) was ruthenium labeled using the BV-Tag™ (BioVeris Europe, Witney, Oxfordshire, UK). This secondary antibody is added to the hPCSK9 coupled beads before measuring in the BioVeris M-SERIES® 384 Analyzer. Sequence analysis of hits from the BioVeris screening is conducted to identify Fab clones. Selected Fab antibodies are sub-cloned into IgG1 format.

Determination of Picomolar Affinities Using Solution Equilibrium Titration (SET)

For K_{D} determination, monomer fractions (at least 90% monomer content, analyzed by analytical SEC; Superdex75, Amersham Pharmacia) of Fab are used. Electrochemiluminescence (ECL) based affinity determination in solution and data evaluation can be performed essentially as described by Haenel et al., 2005. A constant amount of Fab is equilibrated with different concentrations (serial 3″ dilutions) of recombinant hPCSK9 in solution. Biotinylated hPCSK9 coupled to paramagnetic beads (M-280 Streptavidin, Dynal), and BV-Tag™ (BioVeris Europe, Witney, Oxfordshire, UK) labeled anti-human (Fab)'₂ (Dianova) is added and the mixture incubated for 30 min. Subsequently, the concentration of unbound Fab is quantified via ECL detection using the M-SERIES® 384 analyzer (BioVeris Europe).

Affinity determination to PCSK9 of another species (e.g., chimpanzee or cynomolgus) in solution is done essentially as described above, replacing the human PCSK9 with the chimpanzee or cynomolgus PCSK9. For detection of free Fab, biotinylated hPCSK9 coupled to paramagnetic beads is used. Affinities are calculated according to Haenel et al. (2005 Anal Biochem 339, 182-184).

### Example 3

### Generation of Anti-PCSK9 Fab by Phage Display

Anti-PCSK9 Fab was generated using the following phage display techniques. Purified human PCSK9 was labeled with PEO4 biotin (Pierce, 21329) using the manufacturer's protocol using a 20:1 molar ratio of biotin: PCSK9. Low molar ratios ensure limited modification of the protein being labeled and the choice of the PEO4 linker separates the biotin moiety from the protein and enhances the overall hydrophilicity of the biotinylated protein. Biotinylated human PCSK9 was used to coat Dynal M280 streptavidin beads and the Morphosys Hucal library was panned for 3 rounds using standard panning techniques. After three iterative rounds of panning, the pooled round 3 plasmid DNA was purified and digested with restriction enzymes EcoRI and XbaI. Plasmid DNA was separated by agarose gel electrophoresis and the 1.5 kB insert containing two gene segments (immunoglobulin heavy chain (VH/CH) and light chain (VL/CL)) was excised and purified. This 1.5 kB fragment (Fab insert) was subcloned into the Morphosys expression vector pMORPHX9_FH and transformed into electrocompetent TG-1 cells. Individual colonies were picked and master plates were prepared. Daughter plates inoculated from the master plates were re-grown in low glucose media and Fab expression was induced by culture in the presence of IPTG overnight. Cell pellets were frozen, lysed with lysozyme and cleared lysates were evaluated by ELISA on plates coated with PEO-biotinylated PCSK9 coated on neutravidin-coated wells (negative controls neutravidin alone). ELISA positives were retested following restreaking of master plate onto agar plates and picking of 3 individual colonies for retesting. Plasmid DNA from PCSK9 clones was also prepared for DNA sequencing. Fab protein from unique clones was prepared in liter scale cultures induced with IPTG and then purified sequentially by IMAC and size exclusion chromatography. Protein concentrations were determined by Bradford assay coupled with SDS-PAGE.

### Example 4

### PCSK9 Competitive ELISA

Purified human PCSK9 labeled with NHS-PEO4-biotin (Pierce, 21329) was used to coat neutravidin-coated Nunc Maxisorp plates. Following the blocking of non-specific binding with BSA, PCSK9 coated wells were incubated first with a saturating concentration of an anti-human PCSK9 Fab (positive control Fab) or with buffer alone. Following binding of the positive control Fab (or buffer alone), alternate anti-human PCSK9 Fabs (test Fab) were added to both buffer alone and anti-human PCSK9 Fab treated wells. After incubation and wash steps, antibody fragments bound to plate-bound human PCSK9 were detected using a cocktail of peroxidase-conjugated, goat anti-human light chain antibodies with 3,3′,5,5′-tetramethylbenzidine (TMB) substrate. Fabs that compete for similar or overlapping binding sites on human PCSK9 fail to elicit additional binding signals (i.e. binding competition for similar or overlapping sites on human PCSK9) compared to positive control Fab alone. Alternatively, Fabs that bind independently of the positive control Fab exhibit increased binding signals as reflected by increased levels of TMB substrate conversion (i.e. non-competitive binding of Fabs to human PCSK9). Using this strategy, Fabs were grouped based on ability of members to block each others' binding to human PCSK9. Initial characterization with H1-anti-PCSK9-Fab as the positive control Fab divided the antibodies into two groups: inhibited by H1 (group 1) or not inhibited by H1 (group 2). Further binding competition experiments demonstrated that Fabs within each group inhibited the binding of other members of that group. From these studies, a third group of Fabs (group 3) was identified by virtue of non-competition with either group 1 or group 2 Fabs. Fab grouping was utilized as a guide to determine which of the anti-PCSK9 Fabs to characterize for binding affinity, ability to disrupt the hPCSK9/LDL-R, and the effects on HepG2 cells. The precise binding site on human PCSK9 of Fabs with desired properties in vitro, such as H1, were then mapped using biophysical techniques such as DXMS, as illustrated in Example 4.

### Example 5

### Functional Analysis of Anti-PCSK9 Fab

In this example, the functional properties of the H1-anti-PCSK9 Fab were examined, including binding affinity, ability to disrupt the hPCSK9/LDL-R, and the effects on HepG2 cells.

1. Binding Affinity

Biacore binding assays were performed at 25° C. on a T100 instrument. HBS-P+Ca (10 mM HEPES, 150 mM NaCl, pH 7.4, 0.005% P-20, 2 mM CaCl₂) was used as the running buffer. For immobilization, hPCSK9 was diluted in to pH 5.5 acetate buffer at 30 μg/mL right before use. About 200 RU of hPCSK9 was immobilized on a CM5 sensor chip (S Series) following the standard amine coupling protocol. Fab solutions (0-20 nM, diluted in the running buffer) were injected over the PCSK9 and reference surface (blank amine coupling) at a flow rate of 30 μl/min. PCSK9 surface was regenerated with a 60 second injection of 1 mM NaOH and 1 M NaCl.

All data analysis were done using the BIAevluation software. Binding curves were double-reference corrected, first with the binding curve from the reference cell, followed the binding curve from the blank of the running buffer. Then the data was analyzed globally with a 1:1 binding model to extract binding constant K_{D} (nM), association (kₐ, 1/Ms), and dissociation rate constant (k_{d}, 1/s).

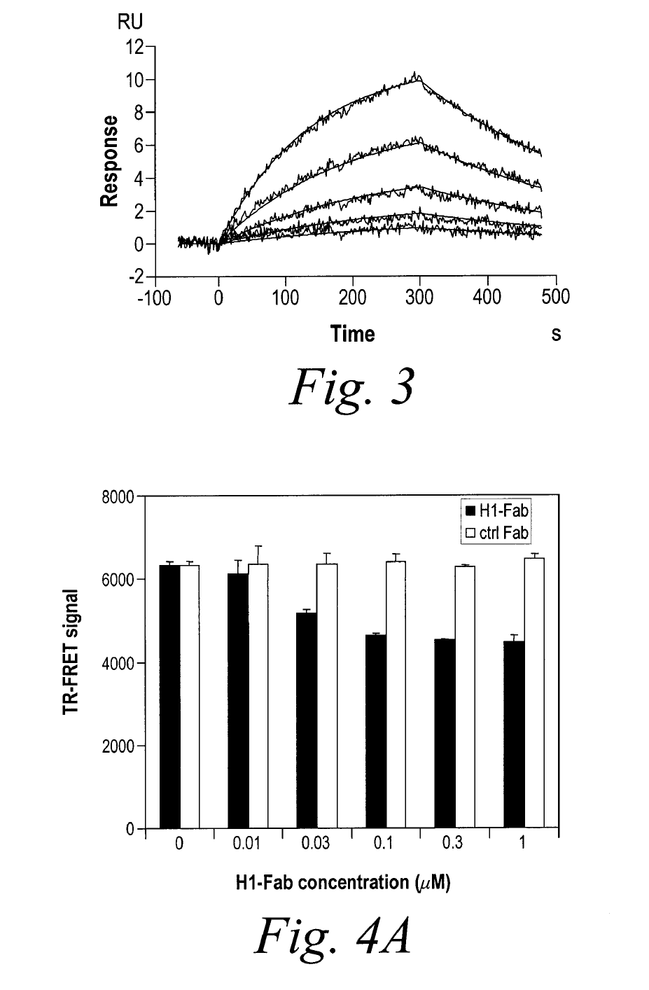

It was determined that H1-Fab exhibited a kₐ of 3.23×10⁵ (1/Ms), k_{d} of 3.41×10⁻³ (1/s), and K_{D} of 1.05×10⁻⁸ M (FIG. 3).

2. Ability of H1-Anti-PCSK9 Fab to Disrupt hPCSK9/LDL-R

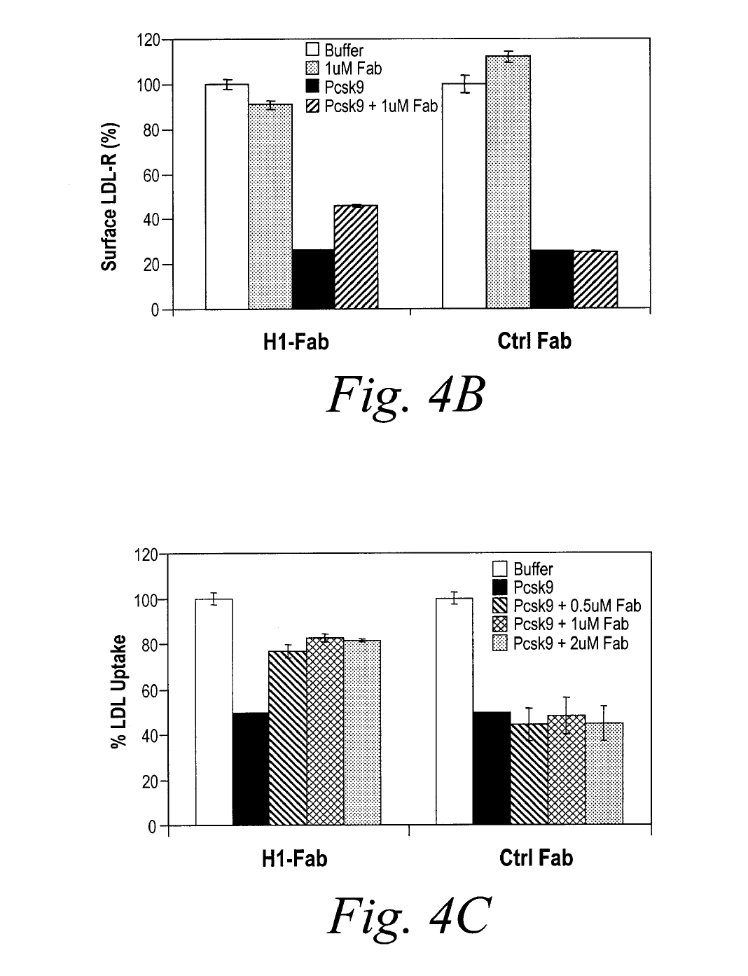

A PCSK9/LDL-R FRET disruption assay was performed as follows to assess the ability of H1-Fab to disrupt the hPCSK9/LDL-R interaction. LDL-R extracellular domain (Ala 22-Arg 788) (R&D Systems) was labeled with europeium cryptate (LDL-R-Eu) (Perkin Elmer) and PCSK9 purified protein was labeled with Alexa Fluor 647 (PCSK9-Alexa) (Invitrogen). The assay buffer consisted of 20 mM HEPES (pH 7.0), 150 mM NaCl, 2 mM CaCl₂, 0.1% Tween 20, and 1 mg/ml BSA. The Fab was pre-incubated with PCSK9-Alexa at room temperature for 30 minutes before LDL-R-Eu was added. The final concentration of PCSK9-ALexa and LDL-R-Eu was 8 nM and 1 nM, respectively. After two hours incubation, the plate was read on Envision (Perkin Elmer) with the following settings: excitation at 330 nm and emissions at both 620 nm and 665 nm, 100 μS delay between excitation and readings. The ratio of reading a 665 nm over reading at 620 nm is normalized and reported in FIG. 4.

As shown in FIG. 4A, H1-Fab disrupted the hPCSK9/LDL-R interaction.

3. Effect on HepG2 Cells

LDL-uptake was measured using flow cytometry. HepG2 cells (ATCC) were maintained in DMEM with 10% (V/V) fetal bovine serum (FBS). Cells were seeded into a 96-well collagen-coated 96-well plate (BD Biosciences), the night before PCSK9 Ab treatment. 200 nM of PCSK9 protein was preincubated with anti-PCSK9 Fab at indicated concentrations for 30 minutes before adding to cells.

After PCSK9 and Fab treatment for 3 hours, dil-LDL (Intracel) was added directly into each well to a final concentration of 5 μg/ml and incubated for one additional hour at 37° C., 5% CO₂. Cells were trypsinized, harvested, and dil-LDL positive cells were measured by flow cytometry (LSR11, BD Biosciences). Geometric means were analyzed using FlowJo 5.7.2 software, normalized to buffer control, and reported in FIG. 4.

Surface LDL was also measured using flow cytometry (FIG. 4B). HepG2 cells were trypsinized, seeded in collagen coated plate and incubated overnight at 37° C. with 5% CO₂ to allow the recovery of LDL-R expression. The following day, PCSK9 protein and PCSK9 Fab were premixed 30 minutes before incubating with cells for 4 hours. Cells were harvested with Versene (Invitrogen) and blocked with donkey serum (Jackson Immunoresearch Laboratories) prior to staining with a rabbit anti-Human LDL-R polyclonal antibody (Fitzgerald) and subsequently with APC-conjugated donkey anti-rabbit IgG antibody (Jackson Immunoresearch Laboratories). After washing, cells were fixed with 2% paraformaldehyde and subjected to flow cytometry analysis on a BD LSR-II cytometer. The averages of geometric means were calculated using FlowJo 5.7.2 software and reported in FIG. 4.

As shown in Figure, H1-Fab led to increased surface LDL-R levels (FIG. 4B) and increased LDL-uptake by Hep2 cells (FIG. 4C).

### Example 6

### Epitope Mapping

In this example, deuterium exchange mass spectrometry (DXMS) was used to determine the epitope(s) recognized by H1-Fab as follows.

A. Materials

Protein diluent (H₂O or D₂O) was 20 mM sodium phosphate, pH 7.3 with 150 mM NaCl. The quenching solution was 0.5% (v/v) trifluoroacetic acid (TFA) in water. All other chemicals were purchased from Sigma, and HPLC grade solvents were from Fisher Scientific. Protein:Fab incubations were prepared and allowed to incubate for at least 2 h at 4° C.

B. Solution Hydrogen/Deuterium (H/D) Exchange

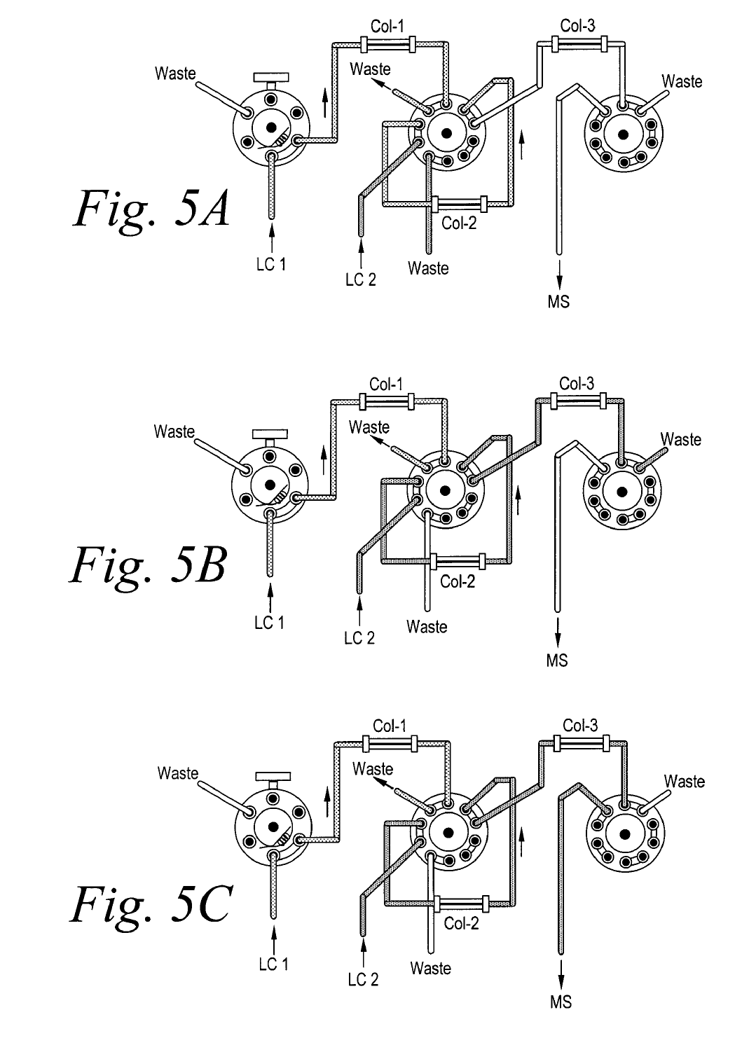

Automated H/D exchange mass spectrometry experiments were performed with a similar setup and similar fashion as described in the literature (Anal. Chem. 2006, 78, 1005-1014). In short, a LEAP Technologies Pal HTS liquid-handler (LEAP Technologies, Carrboro, N.C.) was used for all liquid handling operations. The liquid-handler was controlled by automation scripts written in LEAP Shell that were coded by the manufacturer. The robot was housed in a refrigerated enclosure maintained at 2° C. Plates for sample, diluent, and quench solution were loaded into the liquid-handler trays before the start of an experimental sequence. A 6-port injection valve and a wash station were also mounted on the liquid-handler rail and facilitate sample injection into the chromatographic system and syringe washing, respectively. The chromatographic system consisted of two additional valves, an enzyme column, a reversed-phase trap cartridge, and an analytical column was housed in a separate chamber constructed in house and maintained at 2° C. by peltier stacks. The fluid connections and fitting of the immobilized pepsin, reversed-phase trap cartridge, and analytical column to the valves is illustrated in FIG. 5. Valves and columns were configured in such a way as to allow in-line protein digestion, peptide desalting, and reversed-phase chromatography prior to introduction of the sample into the electrospray ionization (ESI) source of the mass spectrometer. The fluid streams required for operation were provided by two separate Agilent HPLC systems (Agilent 1100, Palo Alto, Calif.). The first HPLC pump (loading pump) delivered 0.05% (v/v) trifluoroacetic acid (TFA) in water at 125 μL/min. The valve positions during the load phase are illustrated in FIG. 5A. In this phase the sample is transferred from the sample loop through the immobilized pepsin cartridge (2 mm×20 mm, kindly provided by Prof. Virgil Woods of UCSD) onto a reversed-phase trap cartridge (1 mm×8 mm, Michrom Bioresources Inc., Auburn, Calif.). Subsequently, auxiliary valve 2 was switched such that the second HPLC pump (gradient pump) delivered a gradient through the reversed phase trap cartridge and the analytical column into auxiliary valve 3. The immobilized enzyme cartridge was isolated to waste in this position. The auxiliary valve 3 was programmed to divert flow to waste for a preset time period for desalting (FIG. 5B) of the sample loaded on the trap cartridge. After the desalting period the valve was switch so as to allow the flow from the gradient pump to reach the ion source of the mass spectrometer (after passing through the trap cartridge and the analytical column, FIG. 5C). The gradient pump delivered a gradient of 0 to 40% mobile phase B over 55 minutes at 50 μL/min (mobile phase A=0.2% formic acid in water, B=0.2% formic acid in acetonitrile).

C. Mass Spectrometry

Liquid Chromatography Electrospray Ionisation Tandem Mass Spectrometry (LC-ESI-MS) was performed on a QT of Ultima Global (Waters, Milford, Mass.) operated in V mode. Two data-dependent MS/MS switching experiments were performed to collect tandem mass spectra for the purpose of identifying the sequences of the peptides generated by on-line proteolysis. Acquisitions performed for the purpose of deuteration level determination were MS-only (5s scans over m/z 400-1500).

D. Complementary Hydrogen/Deuterium (H/D) Exchange Experiments

The protein (hPCSK9 and its prodomain PD) was subjected to several on-and off-exchange conditions with the expected net result being a marking of any potential epitope by an increase in deuteration levels with respect to the control in the protection experiment (described below) and a reduction in deuteration level with respect to the control in the In-D₂O experiment (described below).

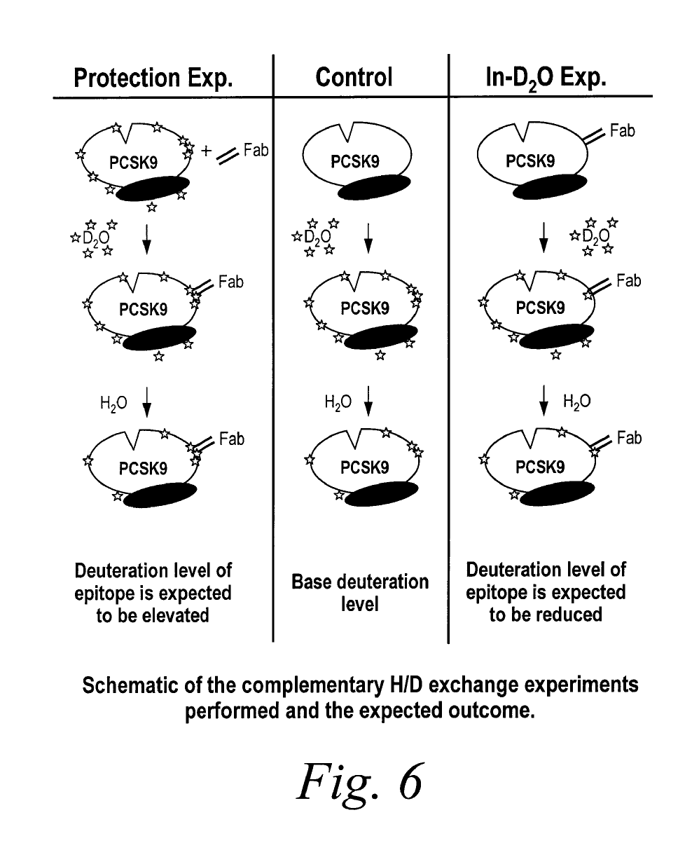

Deuteration, which is the exchange of amide hydrogens on the protein with deuterium is an especially useful tool for the probing of structure and function of proteins because labeling with deuterium does not change structure or function of the labeled protein. Deuterium is an isotope of hydrogen that has twice the mass of hydrogen, indicated in FIG. 6 by stars. This is in stark contrast to other methods of labeling that attach new moieties to existing functional groups on proteins.

1. Protection Experiment

In the protection experiment, deuterated protein soulution was prepared by overnight incubation of the protein in 20 mM sodium phosphate, pH 7.3 with 150 mM NaCl in D₂O. In the control experiment deuterated protein was diluted with H₂O and after varying time periods of off-exchange (e.g. 5 min) quench solution was added. This was followed by on-line digestion with pepsin and LCMS as described above. Off-exchange of the protein:Fab complex was performed by dilution of the deuterated protein solution with an equimolar amount of Fab solution (non-deuterated, see schematic FIG. 6, right column) and incubation for 15 min to form the protein(deuterated):Fab complex. After formation of the complex the sample was treated as described below for the control.

The left column of FIG. 6 illustrates the experimental sequence for the protection experiment, which starts out with deuterated PCSK9. “Deuterated” means that the amide hydrogens of the protein have been replaced with deuterium by incubation of the protein in deuterium buffer for a period of several hours. As illustrated in the second row in the left column of FIG. 6, the binding of the Fab to its epitope on deuterated PCSK9 protein will block part of the surface of PCSK9 around the area of the epitope. The blocking of the surface also reduces solvent access, which is critical for hydrogen/deuterium exchange to occur. In the third row in the left column of FIG. 6 the effect of incubation of deuterated PCSK9/Fab complex in non-deuterated buffer is illustrated.

As shown in FIG. 6, deuteration levels on PCSK9 are rapidly reduced on the solvent accessible areas because H/D exchange can occur freely. In contrast, the reduced solvent access to the area of the epitope due to the blocking action of the Fab that covers the surface causes H/D exchange to be slowed. This results in the preservation of most of the deuteration in the area of the epitope.

It is possible to locate the increased levels of deuteration (marking the epitope) along the protein sequence by cutting of the protein Fab complex into smaller pieces with an enzyme and measuring the deuteration level of each of the fragments with a mass spectrometer. This is possible because of the mass change that results from deuteration as deuterium is heavier than hydrogen. Stitching together the information collected from the fragments allows one to derive the distribution of deuterium across the protein sequence.

2. Control

Because deuteration levels vary greatly across the protein sequence due to protein structure and the resulting variation in solvent accessibility as well as the differences in H/D exchange rates observed for amide bonds formed between different amino acids it is impossible to conclude solely from an elevated deuteration level observed in the protection experiment (described by the left column of FIG. 6) on the presence of an epitope. Fortunately, the natural variation of deuteration levels cancels out in a differential experiment. The differential experiment consists of the measurement of deuteration levels in the presence and absence (control experiment, center column of FIG. 6) of the Fab and calculation of the difference in deuteration. The observed differences in deuteration level are attributable purely to the effects of the Fab and large values will be indicative of the presence and location of epitopes.

3. In-D₂O Experiment

Further, a complementary differential experiment (i.e., an in-D₂O experiment) illustrated in the right column of FIG. 6 can be performed and the expected result deduced using similar reasoning as that put forward for the left column experiment. The major difference being that the observed difference in deuteration for the right column experiment should be opposite in sign to that of the left column and provide therefore complementary evidence for the presence and location of a potential epitope as well as validation of results against each other.

In a typical In-D₂O experiment (see schematic FIG. 6, middle and right column) protein (control) or alternatively protein:Fab complex is diluted into D₂O buffer. After a fixed period of on-exchange the mixture is further diluted with H₂O buffer to cause off-exchange and finally quenched with quench buffer. Once mixed, the quenched solution is fully automatically proteolyzed, separated and analyzed by LCMS as described above. Various D₂O incubation periods (e.g. 45s) were used in the experiment to optimize the observed differences in deuteration between control (protein only) and the protein:Fab sample. The average change in deuteration between sample and control was calculated as the difference between the deuterium uptake levels of the sample and control, where deuterium uptake levels were determined as described below under data processing.

E. Data Processing

Tandem MS acquisitions were reduced to peak lists using MassLynx (Waters, Milford, Mass.) and searched against the protein sequence using Mascot (Matrix Sciences, London, UK). A list of probable peptide sequence identifications returned by the database search were manually validated. MassLynx was used to generate single ion chromatograms of the validated precursor ion masses. Mass spectra of the isotopic distributions of each precursor were summed across the chromatographic peak, smoothed and centered so as to determine the level of deuterium uptake. To assign a deuterium uptake level to each residue of the protein sequence the following procedure was followed. Residues were assigned the normalized deuterium uptake of the peptides that covered them. If more than one peptide covered the same residue the average of the normalized deuterium uptake of all the peptides covering that residue was used. The normalized deuterium uptake for each peptide was calculated by dividing the observed deuteration level by the number of amino acids in that peptide.

F. Results

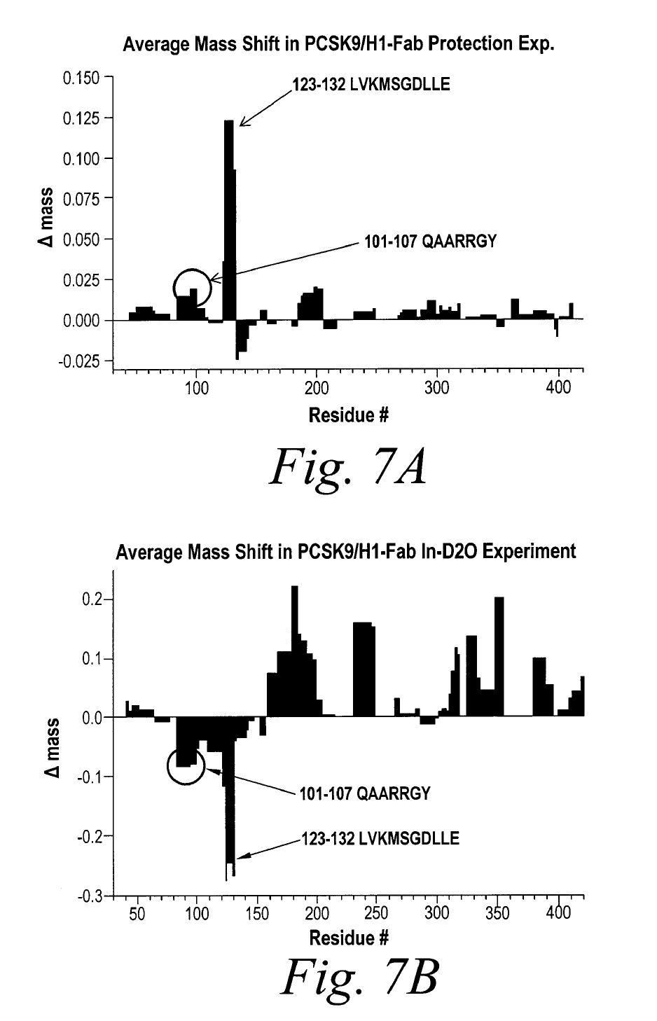

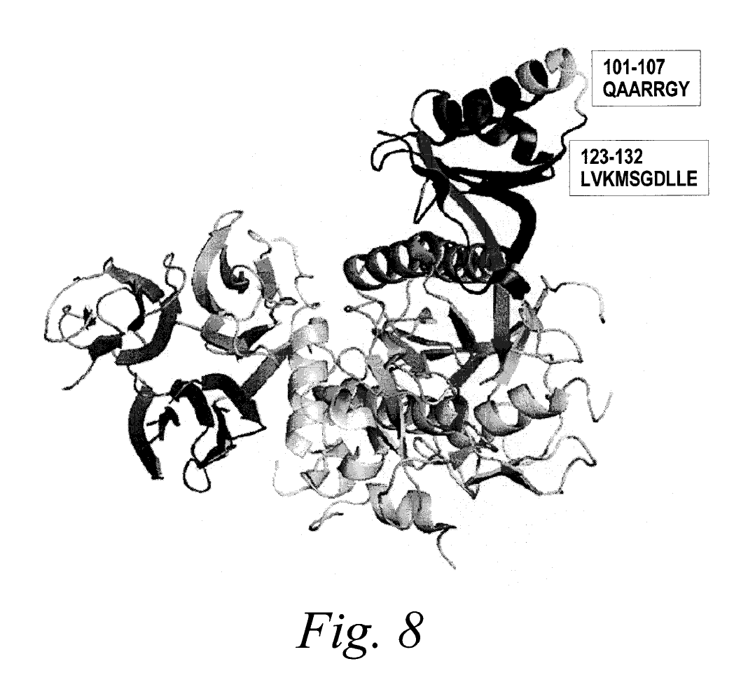

The observed average change in deuteration for the protection and the In-D₂O experiment carried out on hPCSK9 and hPCSK9:H1-Fab complex as a function of residue number of hPCSK9 (prodomain included, cystine rich domain is excluded as it was not covered by the experiments) is shown in FIG. 7. The amino acid sequences of the regions showing the expected behavior of a potential epitope are also shown in FIG. 7.

FIG. 7A shows the change in deuteration for the protection experiment. The change in deuteration is defined as the difference between the deuteration level of the experiment illustrated in FIG. 6 (left column, with Fab present) and its control (middle column, no Fab). The change in deuteration is plotted as the average mass shift per residue over the residue number for amino acid residues 40 to 420 of the PCSK9 sequence (starting with the pro-domain and excluding the cysteine rich domain). A high value for the change in deuteration (indicated as a positive mass shift per residue) is indicative of an epitope. The region of residues 123-132 (sequence annotated in plots) stands out in this regard and is therefore considered to cover all or part of an epitope.

The complementary data from the In-D₂O experiment (illustrated in the right column in FIG. 6) is plotted in FIG. 7A. Comparison of the two plots depicted in FIG. 7 reveals that the stretch of amino acid residues 123-132 LVKMSGDLLE shows the anticipated change of deuteration levels for an epitope as expected from the experimental design (FIG. 6). The region 123-132 (LVKMSGDLLE) in the hPCSK9 crystal structure (see FIG. 8) covers part of a helix and loop and makes physical sense as a potential epitope as it is highly accessible.

Not immediately obvious from the data shown in FIG. 7 is a second region spanning residues 101-107 (QAARRGY) (see FIG. 8), which is a subsection of a larger region that also shows the expected complementary behavior in deuteration levels in FIG. 7 that would be characteristic of a potential epitope. It turns out that the method used for mapping back the change in deuteration level from the deuteration of the observed peptides onto the primary sequence has a strongly smoothing effect, which is desirable as the fluctuations observed in the measurement are quite large.

On the other side, this delocalization of deuterium levels makes it harder to detect the likely participation of the region covered by residue 101-107 in the epitope from the data as it is plotted in FIG. 7. Yet, detailed inspection of the peptides observed to cover the larger region allows most of the observed exchange to be attributed to the much shorter region 101-107.

Further, it is important to note in the crystal structure that this shorter region is spatially located right next to region 123-132, which suggests that both stretches form the non-linear epitope of H1-Fab on hPCSK9. Importantly, the 2 amino acid stretches implicated by the data of FIG. 7 form a non-linear epitope for H1-Fab, which correlates with the predicted SEQ ID NO 2 and 3 amino acid sequences for antigenic epitopes of hPCSK9 (Table 2).

1. An isolated Proprotein convertase subtilisin/kexin type 9 polypeptide (PCSK9) binding molecule comprising an antigen binding portion of an antibody that specifically binds to a PCSK9, wherein the antigen binding portion binds to an epitope within the catalytic domain of human PCSK9 (SEQ ID NO:1) within or overlapping one of the following:
(a) amino acids 166-177 of SEQ ID NO:1;
(b) amino acids 187-202 of SEQ ID NO:1;
(c) amino acids 206-219 of SEQ ID NO:1;
(d) amino acids 231-246 of SEQ ID NO:1;
(e) amino acids 277-283 of SEQ ID NO:1;
(f) amino acids 336-349 of SEQ ID NO:1;
(g) amino acids 368-383 of SEQ ID NO:1; or
(h) amino acids 426-439 of SEQ ID NO:1.

2. An isolated PCSK9 binding molecule comprising an antigen binding portion of an antibody that specifically binds to a PCSK9, wherein the antigen binding portion binds to an epitope within the cysteine-rich domain of human PCSK9 within or overlapping one of the following:
(a) amino acids 443-500 of SEQ ID NO: 1;
(b) amino acids 557-590 of SEQ ID NO: 1; or
(c) amino acids 636-678 of SEQ ID NO: 1.

3. An isolated PCSK9 binding molecule comprising an antigen binding portion of an antibody that specifically binds to a PCSK9, wherein the antigen binding portion binds to an epitope within the pro-domain of human PCSK9 within or overlapping amino acids 89-134 of SEQ ID NO:1.

4. The PCSK9 binding molecule of any of claim 1, wherein the antigen binding portion is cross reactive with a PCSK9 of a non-human primate.

5. The PCSK9 binding molecule of any of claim 1, wherein the antigen binding portion is cross reactive with a PCSK9 of a rodent species.

6. The PCSK9 binding molecule of any of claim 1, wherein the antigen binding portion binds to a linear epitope.

7. The PCSK9 binding molecule of any of claim 1, wherein the antigen binding portion binds to a non-linear epitope.

8. The PCSK9 binding molecule of claim 7, wherein the antigen binding portion binds to a non-linear epitope consisting of at least one portion of each of the following linear epitopes:
(a) amino acids 89-101 of SEQ ID NO:1; and
(b) amino acids 106-134 of SEQ ID NO:1.

9. The PCSK9 binding molecule of claim 7, wherein the antigen binding portion binds to a non-linear epitope consisting of at least one portion of each of the following linear epitopes:
(a) amino acids 166-177 of SEQ ID NO:1; and
(b) amino acids 443-458 of SEQ ID NO:1.

10. The PCSK9 binding molecule of claim 7, wherein the antigen binding portion binds to a non-linear epitope consisting of at least one portion of two or three of the following linear epitopes:
(a) amino acids 187-202 of SEQ ID NO:1;
(b) amino acids 231-246 of SEQ ID NO:1; and
(c) amino acids 368-383 of SEQ ID NO:1.

11. The PCSK9 binding molecule of claim 7, wherein the antigen binding portion binds to a non-linear epitope consisting of at least one portion of each of the following linear epitopes:
(a) amino acids 206-219 of SEQ ID NO:1; and
(b) amino acids 277-283 of SEQ ID NO:1.

12. The PCSK9 binding molecule of claim 7, wherein the antigen binding portion binds to a non-linear epitope consisting of at least one portion of each of the following linear epitopes:
(a) amino acids 336-349 of SEQ ID NO:1; and
(b) amino acids 426-439 of SEQ ID NO:1.

13. The PCSK9 binding molecule of claim 7, wherein the antigen binding portion binds to a non-linear epitope consisting of at least one portion of two or three of the following linear epitopes:
(a) amino acids 459-476 of SEQ ID NO:1;
(b) amino acids 486-500 of SEQ ID NO:1; and
(c) amino acids 557-573 of SEQ ID NO:1.

14. The PCSK9 binding molecule of claim 7, wherein the antigen binding portion binds to a non-linear epitope consisting of at least one portion of two or three of the following linear epitopes:
(a) amino acids 577-590 of SEQ ID NO:1;
(b) amino acids 636-645 of SEQ ID NO:1; and
(c) amino acids 659-677 of SEQ ID NO:1.

15. The PCSK9 binding molecule of claim 2, wherein the antigen binding portion specifically binds to an epitope of human PCSK9 within or overlapping within or overlapping one of the following:
(a) amino acids 443-458 of SEQ ID NO:1;
(b) amino acids 459-476 of SEQ ID NO:1;
(c) amino acids 486-500 of SEQ ID NO:1;
(d) amino acids 557-573 of SEQ ID NO:1;
(e) amino acids 577-590 of SEQ ID NO:1;
(f) amino acids 636-645 of SEQ ID NO:1; or
(g) amino acids 659-677 of SEQ ID NO:1.

16. The PCSK9 binding molecule of claim 3, wherein the antigen binding portion specifically binds to an epitope of human PCSK9 within or overlapping one of the following:
(a) amino acids 89-101 of SEQ ID NO:1; or
(b) amino acids 106-134 of SEQ ID NO:1.

17. The PCSK9 binding molecule of any preceding claim, wherein the antigen binding portion binds to PCSK9 with a dissociation constant (Kₒ) equal to or less than 10 nM.

18. The PCSK9 binding molecule of any preceding claim, wherein the antigen binding portion binds to PCSK9 with a dissociation constant (K₀) equal to or less than 1 nM.

19. The PCSK9 binding molecule of claim 18, wherein the antigen binding portion binds to PCSK9 with a K_{D} equal to or less than 0.5 nM.

20. The PCSK9 binding molecule of claim 19, wherein the antigen binding portion binds to a human PCSK9 with a K₀ equal to or less than 0.1 nM.

21. The PCSK9 binding molecule of claim 18, wherein the antigen binding portion binds to PCSK9 of a non-human primate with a Kₒ equal to or less than 0.3 nM.

22. The PCSK9 binding molecule of claim 18, wherein the antigen binding portion thereof binds to mouse PCSK9 with a Kₒ equal to or less than 0.5 nM.

23. The PCSK9 binding molecule of any preceding claim, wherein the antigen binding portion is an antigen binding portion of a human antibody.

24. The PCSK9 binding molecule of claim 23, wherein the antibody is a humanized or humaneered antibody.

25. The PCSK9 binding molecule of any preceding claim, wherein the antigen binding portion is an antigen binding portion of a monoclonal antibody.

26. The PCSK9 binding molecule of claim 23, wherein the antigen binding portion is an antigen binding portion of a polyclonal antibody.

27. The PCSK9 binding molecule of claim 1, wherein the PCSK9 binding molecule is a chimeric antibody.

28. The PCSK9 binding molecule claim 1, wherein the PCSK9 binding molecule comprises an Fab fragment, an Fab′ fragment, an F(ab′)₂, or an Fv fragment of the antibody.

29. The PCSK9 binding molecule of claim 1, wherein the PCSK9 binding molecule comprises a single chain Fv.

30. The PCSK9 binding molecule of claim 1, wherein the PCSK9 binding molecule comprises a diabody.

31. The PCSK9 binding molecule of any preceding claim, wherein the antigen binding portion is derived from an antibody of one of the following isotypes: IgG1, IgG2, IgG3 or IgG4.

32. The PCSK9 binding molecule of any preceding claim, wherein the PCSK9 binding molecule inhibits PCSK9 binding to a PCSK9 ligand.

33. The PCSK9 binding molecule of any preceding claim, wherein the PCSK9 binding molecule inhibits PCSK9 binding to a low density lipoprotein receptor (LDL-R).

34. The PCSK9 binding molecule of any preceding claim, wherein the PCSK9 binding molecule inhibits proteolytic activity of PCSK9.

35. The PCSK9 binding molecule of claim 34, wherein the PCSK9 binding molecule inhibits proteolysis of the PCSK9 pro-domain.

36. The PCSK9 binding molecule of any preceding claim, wherein the PCSK9 binding molecule inhibits a PCSK9-dependent decrease of LDL-R on a hepatocyte.

37. The PCSK9 binding molecule of claim 36, wherein the PCSK9 binding molecule inhibits PCSK9 dependent degradation of LDL-R on hepatocytes.

38. The PCSK9 binding molecule of any preceding claim, wherein the PCSK9 binding molecule, when contacted with a hepatocyte under conditions in which PCSK9 is present, increases low density lipoprotein cholesterol (LDL-c) uptake by the hepatocyte, relative to LDL-c uptake by a hepatocyte in the absence of the PCSK9 binding molecule.

39. The PCSK9 binding molecule of any preceding claim, wherein the PCSK9 binding molecule binds to PCSK9 in the presence of LDL-C.

40. The PCSK9 binding molecule of any preceding claim, wherein the PCSK9 binding molecule binds to PCSK9 in the presence of serum.

41. A PCSK9 binding molecule comprising a PCSK9 binding domain, wherein the amino acid sequence of the PCSK9 binding domain is at least 75% identical to an amino acid sequence of an immunoglobulin-like fold of a fibronectin, a cytokine receptor, or a cadherin, and wherein the amino acid sequence of the PCSK9 binding domain is altered, relative to the amino acid sequence of the immunoglobulin-like fold, such that the PCSK9 binding domain specifically binds to the PCSK9.

42. The PCSK9 binding molecule of claim 41, wherein the PCSK9 binding domain binds to the PCSK9 with a Kₒ equal to or less than 10 nM.

43. The PCSK9 binding molecule of claim 41, wherein the PCSK9 binding domain binds to the PCSK9 with a Kₒ equal to or less than 1 nM.

44. The PCSK9 binding molecule of claim 41, wherein the Ig-like fold is an Ig-like fold of a fibronectin.

45. The PCSK9 binding molecule of claim 44, wherein the Ig-like fold is an Ig-like fold of fibronectin type III.

46. A pharmaceutical composition comprising the PCSK9 binding molecule of claim 1.

47. A method of increasing LDL-R levels on a hepatocyte, the method comprising contacting the hepatocyte with a PCSK9 binding molecule.

48. A method of increasing LDL-c uptake by a hepatocyte, the method comprising contacting the hepatocyte with a PCSK9 binding molecule, thereby reducing downregulation of LDL-R by PCSK9 and increasing LDL-c uptake by the hepatocyte.

49. A peptide consisting of an amino acid sequence at least 90% identical to one of following amino acid sequences:

| | |
|---|---|
| YRADEYQPPDGG; | (SEQ ID NO: 4) |
| TSIQSDHREIEGRVMV; | (SEQ ID NO: 5) |
| ENVPEEDGTRFHRQ; | (SEQ ID NO: 6) |
| AGVVSGRDAGVAKGAS; | (SEQ ID NO: 7) |
| VQPVGPL; | (SEQ ID NO: 8) |
| VGATNAQDQPVTLG; | (SEQ ID NO: 9) |
| IIGASSDCSTCFVSQS; | (SEQ ID NO: 10) |
| EAWFPEDQRVLTPN; | (SEQ ID NO: 11) |
| ALPPSTHGAGWQLFCR; | (SEQ ID NO: 12) |
| TVWSAHSGPTRMATAIAR; | (SEQ ID NO: 13) |
| CSSFSRSGKRRGERM; | (SEQ ID NO: 14) |
| HVLTGCSSHWEVEDLGT; | (SEQ ID NO: 15) |
| PVLRPRGQPNQCVG; | (SEQ ID NO: 16) |
| SALPGTSHVL; | (SEQ ID NO: 17) |
| RDVSTTGSTSEEAVTAVAI; | (SEQ ID NO: 18) |
| SQSERTARRLQAQ; | (SEQ ID NO: 2) |
| or | |
| GYLTKILHVFHGLLPGFLVKMSGDLLELA. | (SEQ ID NO: 3) |

50. A method of modulating PCSK9 activity in a subject, the method comprising administering to the subject a PCSK9 binding molecule that modulates a biological activity of the PCSK9, wherein the PCSK9 binding molecule exhibits one or more of the following activities:
(a) inhibiting PCSK9 binding to a LDL-R,
(b) inhibiting proteolytic activity of the PCSK9,
(c) inhibiting PCSK9 dependent decrease of LDL-R on a hepatocyte, and
(d) inhibiting PCSK9 dependent degradation of LDL-R in hepatocyte cells.

51. A method of reducing a plasma cholesterol a subject, the method comprising administering to the subject the composition of claim 46 in an amount effective to reduce plasma cholesterol in the subject.

52. The method of claim 51, wherein the amount is effective to reduce LDL-c.

53. The method of claim 52, wherein the subjects concentration of plasma LDL-c is reduced by at least 5%, relative to plasma LDL-c prior to administering the composition.

54. The method of claim 51, wherein the subject is also receiving therapy with a second cholesterol-reducing agent.

55. The method of claim 54, wherein the second cholesterol reducing agent is a statin.

56. The method of claim 51, wherein the subject has, or is at risk for, a lipid disorder.

57. The method of claim 56, wherein the subject is hypercholesterolemic or is at risk for hypercholesterolemia.

58. The method of claim 51, wherein the subject has, or is at risk for, atherosclerosis.

59. The method of claim 51, wherein the subject has, or is at risk for, a cardiovascular disorder.

60. The method of claim 51, wherein the subject is statin-intolerant.

61. The method of claim 51, wherein the subject is resistant to statin therapy.

62. The method of claim 51, wherein, prior to administration of the composition, the subject's total plasma cholesterol level is 200 mg/dl or greater.

63. The method of claim 51, wherein prior to administration of the composition, the subject's plasma LDL-c level is 160 mg/dl or greater.

64. The method of claim 51, wherein the composition is administered intravenously.

65. An isolated PCSK9 binding molecule comprising an antigen binding portion of an antibody that specifically binds to a PCSK9, wherein the antigen binding portion binds to an epitope within the pro-domain of human PCSK9 within or overlapping one of the following:
(a) amino acids 101-107 of SEQ ID NO: 1; or
(b) amino acids 123-132 of SEQ ID NO: 1.

66. The isolated PCSK9 binding molecule of claim 65, wherein the antigen binding portion binds to an epitope within the pro-domain of human PCSK9 within or overlapping amino acids 101-107 of SEQ ID NO:1.

67. The isolated PCSK9 binding molecule of claim 65, wherein the antigen binding portion binds to an epitope within the pro-domain of human PCSK9 within or overlapping amino acids 123-132 of SEQ ID NO:1.

68. An isolated PCSK9 binding molecule that cross-competes for binding to PCSK9 with a PCSK9 binding molecule that binds to an epitope within the pro-domain of human PCSK9 within or overlapping one of the following:
(a) amino acids 101-107 of SEQ ID NO: 1; or
(b) amino acids 123-132 of SEQ ID NO: 1.

69. The PCSK9 binding molecule of claim 65, wherein the antigen binding portion binds to a non-linear epitope.

70. The PCSK9 binding molecule of claim 69, wherein the antigen binding portion binds to a non-linear epitope comprising all or at least a portion of each of the following linear epitopes:
(a) amino acids 101-107 of SEQ ID NO:1; and
(b) amino acids 123-132 of SEQ ID NO:1.

71. An isolated PCSK9 binding molecule comprising an antigen binding portion of an antibody that specifically binds to a PCSK9, wherein the antigen binding portion binds within amino acids 101-132 of SEQ ID NO:1.

72. The isolated PCSK9 binding molecule of claim 71, wherein the antigen binding portion binds within amino acids 101-132 of SEQ ID NO:1 and comprises at least one amino acid from SEQ ID NO:2 and at least one amino acid from SEQ ID NO:3.

73. An isolated PCSK9 binding molecule comprising an antigen binding portion of an antibody that specifically binds to a PCSK9, wherein the antigen binding portion binds to an epitope that overlays at least one amino acid from SEQ ID NO:2 and at least one amino acid from SEQ ID NO:3.

74. An isolated PCSK9 binding molecule comprising an antigen binding portion of an antibody that specifically binds to a PCSK9, wherein the antigen binding portion binds to an epitope selected from the group consisting of an epitope within SEQ ID NO:2, an epitope within SEQ ID NO:3, or an epitope that overlaps at least one amino acid from SEQ ID NO:2 and at least one amino acid from SEQ ID NO:3.

75. The isolated PCSK9 binding molecule of claim 73 where the amino acid of SEQ ID NO:2 is glutamine.

76. The isolated PCSK9 binding molecule of claim 73 where the antigen binding portion overlaps at least two amino acids from SEQ ID NO:3.

77. The isolated PCSK9 binding molecule of claim 76 where the amino acids are glycine and tryrosine.

78. The PCSK9 binding molecule of claim 65, wherein the antibody is a human, humanized, humaneered, or chimeric antibody.

79. The PCSK9 binding molecule of claim 65, wherein the antigen binding portion is an antigen binding portion of a monoclonal or polyclonal antibody.

80. The PCSK9 binding molecule of claim 65, wherein the PCSK9 binding molecule comprises an Fab fragment, a single chain Fv, an Fab′ fragment, an F(ab′)₂, a diabody, or an Fv fragment of the antibody.

81. The PCSK9 binding molecule of claim 65, wherein the antigen binding portion is derived from an antibody of one of the following isotypes: IgG1, IgG2, IgG3 or IgG4.

82. Use of a PSCK9 binding molecule of any of the preceding claims to prepare a medicament for the treatment of disease associated with high cholesterol levels.


SEQUENCE LISTING (18 sequences)

SEQ ID NO: 1 - protein, 692 aa, Homo Sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 2 - protein, 13 aa, Homo Sapiens
SQSERTARRLQAQ

SEQ ID NO: 3 - protein, 29 aa, Homo Sapiens
GYLTKILHVFHGLLPGFLVKMSGDLLELA

SEQ ID NO: 4 - protein, 12 aa, Homo Sapiens
YRADEYQPPDGG

SEQ ID NO: 5 - protein, 16 aa, Homo Sapiens
TSIQSDHREIEGRVMV

SEQ ID NO: 6 - protein, 14 aa, Homo Sapiens
ENVPEEDGTRFHRQ

SEQ ID NO: 7 - protein, 16 aa, Homo Sapiens
AGVVSGRDAGVAKGAS

SEQ ID NO: 8 - protein, 7 aa, Homo Sapiens
VQPVGPL

SEQ ID NO: 9 - protein, 14 aa, Homo Sapiens
VGATNAQDQPVTLG

SEQ ID NO: 10 - protein, 16 aa, Homo Sapiens
IIGASSDCSTCFVSQS

SEQ ID NO: 11 - protein, 14 aa, Homo Sapiens
EAWFPEDQRVLTPN

SEQ ID NO: 12 - protein, 16 aa, Homo Sapiens
ALPPSTHGAGWQLFCR

SEQ ID NO: 13 - protein, 18 aa, Homo Sapiens
TVWSAHSGPTRMATAIAR

SEQ ID NO: 14 - protein, 15 aa, Homo Sapiens
CSSFSRSGKRRGERM

SEQ ID NO: 15 - protein, 17 aa, Homo Sapiens
HVLTGCSSHWEVEDLGT

SEQ ID NO: 16 - protein, 14 aa, Homo Sapiens
PVLRPRGQPNQCVG

SEQ ID NO: 17 - protein, 10 aa, Homo Sapiens
SALPGTSHVL

SEQ ID NO: 18 - protein, 19 aa, Homo Sapiens
RDVSTTGSTSEEAVTAVAI